(19) United States
(12) Patent Application Publication
(10) Pub. No.: US 2026/0356778 A1
(43) Pub. Date: Nov. 12, 2026

(54) METHODS FOR PRODUCING 1,1,2-TRIFLUOROETHANE (HFC-143) and CHLOROTRIFLUOROETHYLENE (CTFE or CFO-1113)

(71) Applicant: Solstice Advanced Materials US, Inc., Morris Plains, NJ (US)

(72) Inventors: Haluk Kopkalli, Staten Island, NY (US); Alex C. Fattore, Charlotte, NC (US); Alicia Ridge, Charlotte, NC (US); Jeremy Norton, Charlotte, NC (US)

(21) Appl. No.: 19/662,603

(22) Filed: Apr. 29, 2026

Related U.S. Application Data
(60) Provisional application No. 63/799,235, filed on May 2, 2025.

(51) Int. Cl. C07C 17/25; B01J 21/18; B01J 23/44; B01J 27/10; B01J 27/138; B01J 27/224

(52) U.S. Cl. CPC C07C 17/25; B01J 21/18; B01J 23/44; B01J 27/10; B01J 27/138; B01J 27/224

(57) ABSTRACT

Methods for producing both 1,1,2-trifluoroethane (HFC-143) and chlorotrifluoroethylene (CTFE or CFO-1113) by the hydrogenation of 1,1,2-trichloro-1,2,2-trifluoroethane (CFC-113) are included. For example, through an integrated two-reactor system that enables production of CTFE and HFC-143 separately, or in a swing or co-production process having concurrent or alternative pathways, one pathway for predominantly producing CTFE, and another pathway for predominantly producing HFC-143 are included.

### CROSS-REFERENCE TO RELATED APPLICATION

This application claims priority to U.S. Provisional Application No. 63/799,235 filed May 2, 2025, which is herein incorporated by reference in its entirety.

### FIELD

The present disclosure relates generally to a method for producing 1,1,2-trifluoroethane (HFC-143) and/or chlorotrifluoroethylene (CTFE or CFO-1113) from 1,1,2-trichloro-1,2,2-trifluoroethane (CFC-113), and more specifically to methods that enable producing HFC-143 and/or CTFE either concurrently or separately.

### BACKGROUND

Chlorotrifluoroethylene (CTFE or CFO-1113) and 1,1,2-trifluoroethane (HFC-143) are commercially important compounds, including for use in the production of HFO-1132, which has recently found increased utility for a variety of uses. HFO-1132 may exist as a mixture of two geometric isomers, the E- or trans isomer and the Z- or cis isomer, which may be used separately or together in various proportions.

Potential end use applications of HFO-1132 include refrigerants, either used alone or in blends with other components, solvents for organic materials, and as a chemical intermediate in the synthesis of other halogenated hydrocarbon solvents.

### SUMMARY

The present disclosure provides methods for producing both 1,1,2-trifluoroethane (HFC-143) and chlorotrifluoroethylene (CTFE or CFO-1113) by the hydrogenation of 1,1,2-trichloro-1,2,2-trifluoroethane (CFC-113), for example, through an integrated two-reactor system that enables production of CTFE and HFC-143 separately, or in a swing or co-production process having concurrent or alternative pathways, one pathway for predominantly producing CTFE, and another pathway for predominantly producing HFC-143.

In one form thereof, a method for producing chlorotrifluoroethylene (CTFE) and 1,1,2-trifluoroethane (HFC-143), comprises reacting 1,1,2-trichloro-1,2,2-trifluoroethane (CFC-113) with hydrogen in the presence of a first catalyst at a temperature from about 450 °C to about 600 °C to produce a first product composition comprising CTFE and CFC-113; separating at least some of the CTFE from the product composition; and further reacting the product composition comprising CFC-113 with hydrogen in the presence of a second catalyst at a temperature of from 150 °C to 400 °C to produce a second product composition comprising HFC-143.

In another form thereof, a method for producing chlorotrifluoroethylene (CTFE) and 1,1,2-trifluoroethane (HFC-143), comprises reacting 1,1,2-trichloro-1,2,2-trifluoroethane (CFC-113) with hydrogen in the presence of a first catalyst at a temperature from about 150 °C to 400 °C to produce a first product composition comprising 1,2-dichloro-1,1,2-trifluoroethane (HCFC-123a); and at least one of: reacting HCFC-123a with hydrogen in the presence of a second catalyst at a temperature from about 150 °C to 400 °C according to a first reaction pathway to produce a second product composition comprising 1,1,2-trifluoroethane (HFC-143); and reacting HCFC-123a in a vapor phase or a liquid phase dehydrochlorination reaction according to a second reaction pathway to produce a third product composition comprising CTFE.

### BRIEF DESCRIPTION OF THE DRAWINGS

FIG. 1 is a schematic diagram of an integrated process layout for the conversion of 1,1,2-trichloro-1,2,2-trifluoroethane (CFC-113) to 1,1,2-trifluoroethane (HFC-143) and chlorotrifluoroethylene (CTFE).

FIGS. 2a and 2b are schematic diagrams of swing or co-production process layouts for the conversion of 1,1,2-trichloro-1,2,2-trifluoroethane (CFC-113) to 1,1,2-trifluoroethane (HFC-143) and chlorotrifluoroethylene (CTFE).

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” or “any range using any two of the foregoing values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value. For example, a range of as low as 1, 2, or 3, or as high as 8, 9, or 10 followed by this phrase encompasses ranges including 1 to 10, or 2 to 8, or 3 to 9.

In the Examples below, the designation “R” is used in connection with the various fluorine-containing refrigerant molecules described herein, for example, “R-143” refers to 1,1,2-trifluoroethane (HFC-143).

As used herein, “CTFE”, “CFO-1113”, “R-1113” are used interchangeably to refer to chlorotrifluoroethylene (CF₂═CFCl).

As used herein, the phrase “based on total moles of organic components of the composition” refers only to carbon-containing components and does not include or encompass non-carbon-containing components including but not limited to hydrogen (H₂), hydrogen chloride (HCl) or hydrogen fluoride (HF).

As used herein, conversion of a reactant molecule (molecule X) during a reaction is calculated using the following equation:

% conversion of molecule X=(moles of molecule X in the feed mixture−moles of molecule X in the product mixture)/(moles of molecule X in the feed mixture)×100

As used herein, selectivity to a molecule formed during a reaction (molecule Y) from a reactant molecule (molecule X) is calculated using the following equation:

% selectivity to molecule Y=(moles of molecule Y in the product mixture−moles of molecule Y in the feed mixture)/(moles of molecule X in the feed mixture−moles of molecule X in the product mixture)×100

### II. Overview

The present disclosure provides a method for producing 1,1,2-trifluoroethane (HFC-143) and chlorotrifluoroethylene (CTFE) from 1,1,2-trichloro-1,2,2-trifluoroethane (CFC-113) in an integrated process according to a two-reaction sequence shown below (“Process 1”), which includes the following two reactions: (i) hydrogenating CFC-113 to produce chlorotrifluoroethylene (CTFE), and (ii) hydrogenating CFC-113 to produce 1,1,2-trifluoroethane (HFC-143).

Schematic equations for the two reactions, Reactions (i) and Reaction (ii), of Process 1 are represented below:

### Process 1

CFCl₂—CF₂Cl (CFC-113)+H₂→CF₂═CFCl (CTFE)+2HCl (i)

CFCl₂—CF₂Cl (CFC-113)+3H₂→CFH₂—CF₂H (HFC-143)+3HCl (ii)

Step (i) may proceed through an integrated two-reactor system, wherein the first reaction to produce CTFE takes place in the first reactor, and the product composition from the first reactor is then processed (e.g., separating CTFE and recovering unreacted CFC-113 and/or H₂) before proceeding through the second reactor to produce HFC-143. The integrated two-reactor system advantageously enables production of CTFE and HFC-143 separately or simultaneously.

The present disclosure provides a method for producing 1,1,2-trifluoroethane (HFC-143) and chlorotrifluoroethylene (CTFE) from 1,1,2-trichloro-1,2,2-trifluoroethane (CFC-113) in a swing or co-production process having two pathways, one pathway for predominantly producing CTFE, and another pathway for predominantly producing HFC-143. The first pathway for producing HFC-143 operates according to a two-step process shown below (“Process 2—Pathway I”), and the second pathway for producing CTFE operates according to a two-step process shown below (“Process 2—Pathway II”). Each pathway of Process 2 produces 1,2-dichloro-1,1,2-trifluoroethane (HCFC-123a) as an intermediate (e.g., main product in step (i)) of the two-step process. Depending on the desired end product (e.g., predominantly CTFE, predominantly HFC-143, or both CTFE and HFC-143), the swing or co-production process is configured to direct a fraction of the HCFC-123a produced in Step (i) of Process 2 to go through Pathway I, Pathway II, or both Pathway I and Pathway II. For example, 100% of the HCFC-123a produced in Step (i) of Process 2 may be directed towards Pathway I if the desired product is HFC-143. For example, 40% of the HCFC-123a produced in Step (i) of Process 2 may be directed towards Pathway I while the other 60% is directed towards Pathway II if the product demand is 40 mol % HFC-143 and 60 mol % CTFE.

Schematic equations for the two steps (“Step (i)” and “Step (ii)”) of Process 2—Pathway I is represented below:

### Process 2—Pathway I

CFCl₂—CF₂Cl (CFC-113)+H₂→CHClF—CClF₂ (HCFC-123a)+HCl (i)

CHClF—CClF₂ (HCFC-123a)+2H₂→CFH₂—CF₂H (HFC-143)+2HCl (ii)

Schematic equations for the two steps (“Step (i)”, “Step (ii) Vapor Phase”, and “Step (ii) Liquid Phase”) of Process 2—Pathway II is represented below: Process 2—Pathway II

CFCl₂—CF₂Cl (CFC-113)+H₂→CHClF—CClF₂ (HCFC-123a)+HCl (i)

(vapor phase) CHClF—CClF₂ (HCFC-123a)→CFCl═CF₂ (CTFE)+HCl (ii)-(a)

(liquid phase) CHClF—CClF₂ (HCFC-123a)+MOH→CFCl═CF₂ (CTFE)+MCl+H₂O (M=K, Na, or Ca) (ii)-(b)

The present disclosure provides a method for producing E-1,2-difluoroethylene (HFO-1132E) from 1,1,2-trifluoroethane (HFC-143) according to a three-step process shown below (“Process 3”), which includes the following two steps: (i) dehydrofluorinating HFC-143 to produce a mixture of trans-1,2-difluoroethylene (HFO-1132E) and cis-1,2-difluoroethylene (HFO-1132Z), and (ii) optionally isomerizing HFO-1132Z to HFO-1132E.

Schematic equations for the three steps of Process 3 are represented below:

### Process 3

CFH₂—CF₂H (HFC-143)+MOHₙ→trans-CFH═CHF (HFO-1132E)+cis-CFH═CFH (HFO-1132Z)+MFₙ+H₂O (M=K, Na, or Ca; n=1, or 2) (i)

cis-CFH═CFH (HFO-1132Z)→trans-CFH═CHF (HFO-1132E) (ii)

Further details regarding each of the processes are set forth below.

### III. Process 1

### Process Flow

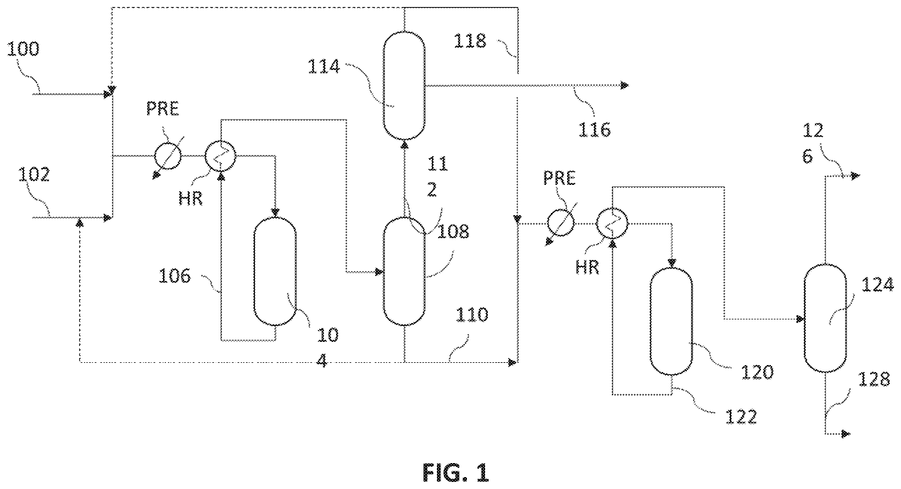

FIG. 1 includes a schematic process flow diagram illustrating suitable components for the reactions in Process 1 converting CFCl₂—CF₂Cl (CFC-113) to CF₂═CFCl (CTFE) and CFH₂—CF₂H (HFC-143).

Referring to FIG. 1, stream 100 comprising hydrogen (H₂) and stream 102 comprising CFC-113 enter reactor 104. Inside reactor 104, the CFC-113 undergoes the hydrogenation reaction, Reaction (i) of Process 1, to produce a first product composition comprising CTFE. Reactor 104 may be pre-loaded with a catalyst for catalyzing the hydrogenation reaction. Stream 106 including first product composition enters separation unit 108 for separating CTFE from byproducts (e.g., trifluoroethene, HCFC-123a, 1-chloro-1,2,2-trifluoroethane (HCFC-133), hydrogen chloride (HCl), etc.) and unreacted reactants (e.g., hydrogen and CFC-113). The separation unit 108 may include one or more of, or a combination of, columns (e.g., distillation columns), scrubbers, adsorbers, absorbers, membrane separation units, flash vessels, vaporizers, etc.

Bottom stream 110 from the separation unit 108 containing predominantly CFC-113 may either be recycled or used as feed for Reaction (ii) of Process 1. Top stream 112 from the separation unit 108 containing hydrogen and CTFE enters a hydrogen recovery unit 114 to produce desired product CTFE in stream 116. Stream 118 including predominantly hydrogen may either be recycled or used as feed for Reaction (ii) of Process 1. Additional feed including CFC-113 and/or hydrogen may be supplied to stream 110 and/or stream 118 to adjust the amount and ratio of reactant composition that enters reactor 120.

The flow of streams 110 and 118 are controlled depending on the desired end product. The control may be, for example, by one or more selective valving arrangements, one or more feed storage tanks, etc. For example, when the desired end product is CTFE, both stream 110 and 118 may be configured to recycle back to reactor 104. When the desired end product includes both CTFE and HFC-143, stream 118 comprising hydrogen (H₂) and stream 110 comprising CFC-113 are configured to enter reactor 120. Inside reactor 120, the CFC-113 undergoes the hydrogenation reaction, Reaction (ii) of Process 1, to produce a second product composition comprising HFC-143. Stream 122 including second product composition enters separation unit 124 (e.g., columns, scrubbers, adsorbers, absorbers, membrane separation units, flash vessels, vaporizers, etc.) for producing stream 126 including predominantly HFC-143 and recycle stream 128.

The hydrogenation reactions of Reaction (i) and Reaction (ii) of Process 1 may be carried out in the gas or vapor phase in a suitable reactor, for example a tubular reactor made from a material which is resistant to temperature and/or corrosion including but not limited to nickel and its alloys, including Hastelloy (for example, Hastelloy C276), Inconel (for example, Inconel 600), Incoloy (for example, Incoloy 825), and Monel (for example, Monel 400), and the vessels may be lined with fluoropolymers.

The reactors (e.g., reactor 104, reactor 120) used for the hydrogenation reaction of Reaction (i) or Reaction (ii) of Process 1, may be first cleaned and flushed with an inert gas including but not limited to nitrogen, followed by packing with a catalyst including but not limited to those described below. The catalyst may be pretreated within the reactor or externally in a separate vessel including but not limited to by drying in the manner described further below, followed by metering the reactants into the reactor to initiate the reaction.

The process flow for each of the Reaction (i) or Reaction (ii) may be in the down or up direction through a bed of the catalyst. Products may be flowed through one or more separation units (e.g., separation unit 108, 124) to remove undesired byproducts from the reaction, including but not limited to hydrogen fluoride (HF) and/or hydrogen chloride (HCl), and the reaction products may be collected in a suitable vessel, for example.

As shown throughout FIG. 1, each reactor (e.g., reactor 104, reactor 120) may optionally be equipped with a pre-heater (denoted by PRE in FIG. 1) to pre-heat the reactants prior to entering the reactor. In addition, each reactor (e.g., reactor 104, reactor 120) may optionally be equipped with a heat recovery (denoted by HR in FIG. 1) unit to cool down hot reactor effluent by heating the incoming reactor feed.

The catalysts and process conditions for each of Reaction (i) and Reaction (ii) in Process 1 are discussed in more detail below.

### Process 1 Reaction (i) Catalysts

In the hydrogenation reaction of Process 1 Reaction (i), the catalyst comprises, consists essentially of, or consists of silicon and/or a nitride, carbide or oxide of silicon. Among the silicon-based catalysts, silicon carbide and silicon nitride are preferred. As used herein, the term silicon carbide refers to all compounds consisting of silicon and carbon. Preferred silicon carbide (carborundum) is available from several sources, including under the trade designation Carborex® by Washington Mills or Advanced Ceramics-Materials GmbH, Germany. As used herein, the term silicon nitride refers to all compounds consisting of silicon and nitrogen. Preferred among the silicon nitrides is Si₃N₄. Preferred silicon nitride is available from several sources, including from UBE Industries, LTD.

### Process 1 Reaction (i) Catalyst Support

In the hydrogenation reaction of Process 1 Reaction (i), the catalyst may be used without a support. Unsupported catalysts used may include silicon-based catalysts, for example, unsupported silicon carbide, unsupported silicon nitride and unsupported silicon oxide.

In the hydrogenation reaction of Process 1 Reaction (i), the catalyst may be supported on a suitable support, including but not limited to carbon. The carbon may be activated carbon. The supported catalyst may be produced by impregnation of any of the suitable supports with a solution of a compound of the desired metal constituent. The support may also be in the form of pellets. After the impregnation step, the solvent may be removed using heat or under vacuum resulting in a solid mass which can be further dried, calcined, and reduced to form active metal catalyst. Supported catalysts used may include silicon-based catalysts, for example, silicon carbide supported on carbon, silicon nitride supported on carbon, and silicon oxide supported on carbon.

In the hydrogenation reaction of Process 1 Reaction (i), when the catalyst is used on a support, catalyst loading on the support is from about 2% to about 25%, or from about 3% to about 25%, or from about 4% to about 25%, or from about 5% to about 25%, or from about 5% to about 24%, or from about 5% to about 23%, or from about 5% to about 22%, or from about 5% to about 21%, or from about 5% to about 20% on the support.

In the hydrogenation reaction of Process 1 Reaction (i), the metal catalysts supported on various catalyst supports used are listed in Table 1 below.

TABLE 1
Metal Catalysts - Process 1 Reaction (i)

| Catalyst | Support |
|---|---|
| Silicon carbide | Activated Carbon |
| Silicon nitride | Activated Carbon |
| Silicon oxide | Activated Carbon |
| Silicon carbide | None |
| Silicon nitride | None |
| Silicon oxide | None |

### Process 1 Reaction (i) Temperature

For reactions using each catalyst/support combination (each row) in Table 1, the reaction temperature of Reaction (i) of Process 1 is as low as about 400 °C, about 450 °C, about 500 °C, or as high as about 550 °C, about 600 °C, about 650 °C, or within any range encompassed by two of the foregoing values as endpoints, for example from about 400 °C to about 650 °C, or from about 450 °C to about 600 °C, for example. The temperature is preferably from about 500 °C to about 600 °C, and more preferably from about 500 °C to about 550 °C.

### Process 1 Reaction (i) Contact Time

For reactions using each catalyst/support combination (each row) in Table 1, the contact time of the reactants with the catalyst (silicon carbide, silicon nitride, silicon oxide) in Reaction (i) of Process 1 is as little as about 0.1 second, about 0.5 second, about 1 second, about 1.5 seconds, about 2 seconds, about 2.5 seconds, about 3 seconds, or as long as about 3.5 seconds, about 4 seconds, about 5 seconds, about 10 seconds, about 15 seconds, about 20 seconds, about 30 seconds, or within any range encompassed by two of the foregoing values as endpoints, for example from about 0.1 seconds to about 30 seconds, from about 1 second to about 10 seconds, or from about 2 seconds to about 5 seconds. For example, the contact time is from about 2.5 second to about 3.5 seconds.

### Process 1 Reaction (i) Pressure

For reactions using each catalyst/support combination (each row) in Table 1, the pressure inside which reactor the Reaction (i) of Process 1 should be such that the reactants are in the vapor phase at the reaction temperature. The pressure inside the reactor for Reaction (i) of Process 1 is as little as about 0.1 psig, about 0.5 psig, about 1 psig, about 3 psig, about 5 psig, about 10 psig, or about 15 psig, or as great as about about 20 psig, about 30 psig, about 35 psig or about 40 psig, or within any range encompassed by two of the foregoing values as endpoints, for example from about 0.1 psig to about 40 psig, or from about 0.5 psig to about 35 psig. For example, the pressure is from about 0.1 psig to about 30 psig.

### Process 1 Reaction (i) Hydrogen Mole Ratio

For reactions using each catalyst/support combination (each row) in Table 1 used in Reaction (i) of Process 1, the mole ratio of hydrogen to CFC-113 is as little as about 1:10, about 1:9, about 1:8, about 1:7, about 1:6, about 1:5, about 1:4, about 1:3, or as great as about 1:2, about 1:1, about 2:1, about 3:1, about 4:1, about 5:1, about 6:1, for example, or within any range encompassed by two of the foregoing values as endpoints. The mole ratio of hydrogen to CFC-113 is preferably about 1:1.

### Process 1 Reaction (i) Selectivity to CTFE

As demonstrated by the Examples herein, for reactions using each catalyst/support combination (each row) in Table 1, the Reaction (i) of Process 1 may achieve a selectivity to the desired product CTFE of at least about 80%, at least about 90%, at least about 95%, at least about 96%, at least about 98%, and for each of the foregoing, less than or equal to 100%, or within any range encompassed by two of the foregoing values as endpoints, for example from about 80% to about 100%, from about 90% to about 100%, or from about 90% to about 98%, based on total moles of the organic components of the composition.

Process 1—Reaction (i) Product Stream from Reactor

Referring to FIG. 1, Reaction (i) of Process 1, the reaction inside reactor 104 produces a product stream 106 (before any post processing steps) comprising target product CTFE, recyclable intermediates including but not limited to HFO-1123, HCFC-123a. and HCFC-133.

For example, for Reaction (i) of Process 1 carried out using each catalyst/support combination (each row) in Table 1, referring to FIG. 1, in the product composition in stream 106 from the reactor 104, the amount of CTFE is at least 90 mol % and less than or equal to 97 mol %, and the amount of HFO-1123 is at least 3 mol % and less than or equal to 5 mol %, based on the combined total moles of CTFE, HFO-1123, HCFC-123a, and HCFC-133 in the product composition in stream 106 from the reactor 104.

The combined total moles of CTFE, HFO-1123, HCFC-123a, and HCFC-133 in the composition set forth in the preceding paragraph is at least 15 mol % and less than or equal to 100% of the total moles of organic components of the product composition in stream 106 from the reactor 104, while the combined amount of other components including unreacted CFC-113 is greater than or equal to 0 mol % and less than 85 mol % of the total moles of organic components of the product composition in stream 106 from the reactor 104.

Process 1 Reaction (i)—Product Stream Composition Post Processing

Referring again to FIG. 1, separation unit 108 may include one or more of columns (e.g., distillation columns), scrubbers, adsorbers, absorbers, membrane separation units, flash vessels, vaporizers, etc. for separating CTFE from byproducts (e.g., trifluoroethane (R-1123), HCFC-123a, 1-chloro-1,2,2-trifluoroethane (HCFC-133), hydrogen chloride (HCl), 2-chloro-1,1-difluoroethylene (R-1122 or HCFC-1122), etc.) and unreacted reactants (e.g., hydrogen and CFC-113).

Bottom stream 110 from the separation unit 108 containing predominantly CFC-113 (e.g., at least 90% CFC-113) may either be recycled or used as feed for Reaction (ii) of Process 1. Top stream 112 from the separation unit 108 containing hydrogen and CTFE enters a hydrogen recovery unit 114 to produce desired product CTFE in stream 116.

For Reaction (i) of Process 1 carried out using each catalyst/support combination (each row) in Table 1, referring to FIG. 1, the amount or purity of CTFE in top stream 112 from separation unit 108 is at least 88 wt %, at least 89 wt %, at least 90 wt %, or at least 91 wt %, and for each of the foregoing, less than or equal to 100 wt %, for example, based on total amount of organic components in the composition.

For Reaction (i) of Process 1 carried out using each catalyst/support combination (each row) in Table 1, referring to FIG. 1, the amount or purity of R-1123 in top stream 112 from separation unit 108, if present, is greater than or equal to 0.1 wt % and yet less than 10 wt %, less than 5 wt %, or less than 1 wt %, for example, based on total amount of organic components of the composition.

For Reaction (i) of Process 1 carried out using each catalyst/support combination (each row) in Table 1, referring to FIG. 1, the amount or purity of R-133 in top stream 112 from separation unit 108, if present, is greater than or equal to 0.1 wt % and yet less than 3 wt %, less than 2 wt %, less than 1 wt %, or less than 0.5 wt %, for example, based on total amount of organic components of the composition.

For Reaction (i) of Process 1 carried out using each catalyst/support combination (each row) in Table 1, referring to FIG. 1, the amount or purity of R-123a in top stream 112 from separation unit 108, if present, is greater than or equal to 0.1 wt % and yet less than 5 wt %, less than 4 wt %, less than 3 wt %, less than 2 wt %, or less than 1 wt %, for example, based on total amount of organic components of the composition.

For example, for Reaction (i) of Process 1 carried out using each catalyst/support combination (each row) in Table 1, referring to FIG. 1, in the final product stream 116 from hydrogen recovery unit 114, the amount of CTFE is at least 90 wt % and less than or equal to 99.95 wt %, the amount of R-1122 is at least 0.01 wt % and less than or equal to 2 wt %, the amount of R-133 is at least 0.01 wt % and less than or equal to 2 wt %, the amount of dimers is at least 0.01 wt % and less than or equal to 2 wt %, the amount of R-123a s at least 0.01 wt % and less than or equal to 2 wt %, and the amount of R-1123 is at least 0.01 wt % and less than or equal to 2 wt %, based on the total amount of organic components of the final product composition in stream 116 from hydrogen recovery unit 114.

For example, for Reaction (i) of Process 1 carried out using each catalyst/support combination (each row) in Table 1, referring to FIG. 1, in the final product stream 116 from hydrogen recovery unit 114, the amount of CTFE is at least 95 wt % and less than or equal to 99.95 wt %, the amount of R-1122 is at least 0.01 wt % and less than or equal to 1 wt %, the amount of R-133 is at least 0.01 wt % and less than or equal to 1 wt %, the amount of dimers is at least 0.01 wt % and less than or equal to 1 wt %, the amount of R-123a is at least 0.01 wt % and less than or equal to 1 wt %, and the amount of R-1123 is at least 0.01 wt % and less than or equal to 1 wt %, based on the total amount of organic components of the final product composition in stream 116 from hydrogen recovery unit 114.

For example, for Reaction (i) of Process 1 carried out using each catalyst/support combination (each row) in Table 1, referring to FIG. 1, in the final product stream 116 from hydrogen recovery unit 114, the amount of CTFE is at least 99.7 wt % and less than or equal to 99.95 wt %, the amount of R-1122 is at least 0.01 wt % and less than or equal to 0.05 wt %, the amount of R-133 is at least 0.01 wt % and less than or equal to 0.05 wt %, the amount of dimers is at least 0.01 wt % and less than or equal to 0.1 wt %, the amount of R-123a is at least 0.01 wt % and less than or equal to 0.05 wt %, and the amount of R-1123 is at least 0.01 wt % and less than or equal to 0.05 wt %, based on the total amount of organic components of the final product composition in stream 116 from hydrogen recovery unit 114.

### Process 1 Reaction (ii) Catalysts

In the hydrogenation reaction of Process 1 Reaction (ii), the catalyst may comprise a metal including but not limited to palladium, platinum, rhodium, ruthenium, iron, cobalt or nickel. Specifically, the catalyst active to catalyze the reaction may preferably be palladium metal (Pd), platinum metal (Pt), or a combination of palladium metal and platinum metal.

### Process 1 Reaction (ii) Catalyst Support

In the hydrogenation reaction of Process 1 Reaction (ii), the catalyst may be supported on a suitable support, including but not limited to carbon or alumina (aluminum oxide—Al₂O₃). The carbon may be activated carbon. The alumina may be alpha(α) alumina, theta(θ) alumina, delta(δ) alumina, or gamma(γ) alumina. The supported catalyst may be produced by impregnation of any of the suitable supports with a solution of a compound of the desired metal constituent. The support may also be in the form of pellets. After the impregnation step, the solvent may be removed using heat or under vacuum resulting in a solid mass which can be further dried, calcined, and reduced to form active metal catalyst.

In the hydrogenation reaction of Process 1 Reaction (ii), the catalyst may be palladium on a carbon support, may be platinum on a carbon support, may be rhodium on a carbon support, and/or may be palladium, platinum, or rhodium on an alumina support. The catalyst may preferably be palladium on a carbon support or palladium on an alpha alumina support.

In the hydrogenation reaction of Process 1 Reaction (ii), the metal catalysts supported on various catalyst supports used are listed in Table 2 below.

TABLE 2
Supported Metal Catalysts - Process 1 Reaction (ii)

| Catalyst | Support |
|---|---|
| Pd | Activated Carbon |
| Pd | alpha(α)-Al₂O₃ |
| Pd | theta(θ)-Al₂O₃ |
| Pd | delta(δ)-Al₂O₃ |
| Pd | gamma(γ)-Al₂O₃ |
| Pt | Activated Carbon |
| Pt | alpha(α)-Al₂O₃ |
| Pt | theta(θ)-Al₂O₃ |
| Pt | delta(δ)-Al₂O₃ |
| Pt | gamma(γ)-Al₂O₃ |
| Rh | Activated Carbon |
| Rh | alpha(α)-Al₂O₃ |
| Rh | theta(θ)-Al₂O₃ |
| Rh | delta(δ)-Al₂O₃ |
| Rh | gamma(γ)-Al₂O₃ |

### Process 1 Reaction (ii) Catalyst Loading

For each catalyst/support combination (each row) in Table 2 used in Reaction (ii) of Process 1, the amount of metal loading on the support is from as little as about 0.01 wt. %, about 0.05 wt. %, about 0.1 wt. %, about 0.2 wt. %, about 0.3 wt. %, about 0.4 wt. %, about 0.5 wt. %, about 1 wt. %, or as great as about 2 wt. %, about 3 wt. %, about 5 wt. %, about 10 wt. %, about 20 wt. %, about 30 wt. %, about 40 wt. %, about 50 wt. %, based on a total weight of the catalyst and support, or within any range encompassed by two of the foregoing values as endpoints, for example, from about 0.01 wt. % to about 20 wt. %, from about 0.01 wt. % to about 10 wt. %, from about 0.1 wt. % to about 10 wt. %, 0.2 wt. % to about 10 wt. %, from about 0.5 wt. % to about 5 wt. %, from about 1 wt. % to about 5 wt. %, from about 1 wt. % to about 4 wt. %, or about 2 wt. % to about 4 wt. %, based on a total weight of the catalyst and support. For supported noble metal catalysts (e.g., Pd or Pt or Rh), the metal loading may be from about 0.01 wt. % to about 5 wt. % based on a total weight of the catalyst and support.

The catalyst loading may be relatively high to encourage conversion of the less reactive intermediates. For example, the Pd loading on a carbon or alpha alumina support is from about 0.1 wt. % to about 10 wt. % palladium metal, for example about 0.1 wt. %, about 0.2 wt. %, about 0.5 wt. %, about 2 wt. %, about 3 wt. %, 4 wt. %, about 5 wt. %, about 6 wt. %, about 7 wt. %, about 8 wt. %, about 9 wt. %, or about 10 wt. % palladium metal, or within any range encompassed by any two of the foregoing values as endpoints, based on a combined weight of the palladium metal and the carbon or alpha alumina support.

### Process 1 Reaction (ii) Catalyst BET Surface Area

The BET (Brunauer, Emmet, and Teller) analysis is the standard method for determining surface areas from nitrogen adsorption isotherms. The BET surface areas of catalysts may be measured using TriStar II Micromeritics instrument. Catalyst samples are degassed using FlowPrep 060 instrument before BET analysis.

For each catalyst/support combination (each row) in Table 2 used in Reaction (ii) of Process 1, the BET surface area may be as low as about 0.5 m²/g, about 1 m²/g, about 3 m²/g, about 5 m²/g, about 10 m²/g, about 15 m²/g, about 20 m²/g 2, about 30 m²/g, about 40 m²/g, about 50 m²/g, about 100 m²/g, about 200 m²/g, or as high as about 250 m²/g, about 300 m²/g, about 400 m²/g, about 500 m²/g, about 600 m²/g, about 700 m²/g m², about 800 m²/g, about 900 m²/g, about 1000 m²/g, about 2000 m²/g, about 3000 m²/g, or within any range encompassed by any of the foregoing values as endpoints, from example, from about 0.5 m²/g to about 3000 m²/g, from about 1 m²/g to about 2000 m²/g, from about 1000 m²/g to about 2000 m²/g, from about 0.5 m²/g to about 500 m²/g, from about 0.5 m²/g to about 300 m²/g, from about 0.5 m²/g to about 5 m²/g, or from about 200 m²/g to about 300 m²/g.

For carbon supported metal catalysts (Pd, Pt, Rh) used in Reaction (ii) of Process 1, the BET surface area may be from about 100 m²/g to about 3000 m²/g, preferably from about 200 m²/g to about 2000 m²/g, more preferably from about 500 m²/g to about 1500 m²/g, and most preferably from about 1000 m²/g to about 1500 m²/g.

For alumina (alpha(α)-Al₂O₃, theta(θ)-Al₂O₃, delta(δ)-Al₂O₃, or gamma(γ)-Al₂O₃) supported metal catalysts (Pd, Pt, Rh) used in Reaction (ii) of Process 1, the BET surface area may be from about 0.5 m²/g to about 500 m²/g, preferably from about 1 m²/g to about 200 m²/g, more preferably from about 1 m²/g to about 100 m²/g, and most preferably from about 1 m²/g to about 20 m²/g.

### Process 1 Reaction (ii) Catalyst Pretreatment

For each catalyst/support combination (each row) in Table 2 used in Reaction (ii) of Process 1, the catalyst may be pretreated by a variety of methods to improve its performance and effectiveness in the reaction. For example, the catalyst may be dried at elevated temperatures, as low as about 200 °C, about 250 °C, about 300 °C, about 350 °C, about 360 °C, about 370 °C, or as high as about 380 °C, about 390 °C, about 400 °C, about 450 °C, about 500 °C, about 600 °C, about 700 °C, or within any range encompassed by two of the foregoing values as endpoints, for example from about 200 °C to about 400 °C, from about 200 °C to about 350 °C, from about 250 °C to about 350 °C, from about 250 °C to about 300 °C, or from about 260 °C to about 300 °C.

For each catalyst/support combination (each row) in Table 2 used in Reaction (ii) of Process 1, as part of the catalyst pretreatment, the catalyst (Pd, Pt, Rh) may be exposed to an inert gas including but not limited to N₂. The pretreatment process may take as low as about 1 hour, about 2 hours, about 3 hours, about 4 hours, or as high about 5 hours, about 6 hours, about 10 hours, about 20 hours, or within any range encompassed by two of the foregoing values as endpoints for example from about 2 hours to about 4 hours, from about 1 hour to about 20 hours, from about 2 hours to about 10 hours, from about 3 hours to about 6 hours, or from about 4 hours to about 5 hours.

### Process 1 Reaction (ii) Temperature

For reactions using each catalyst/support combination (each row) in Table 2, the reaction temperature of Reaction (ii) of Process 1 is as low as about 100 °C, about 125 °C, about 150 °C, about 200 °C, about 205 °C, about 210 °C, about 215 °C, about 220 °C, about 225 °C, about 230 °C, about 235 °C, about 240 °C, about 245 °C, about 250 °C, about 255 °C, or as high as about 260 °C, about 265 °C, about 270 °C, about 275 °C, about 280 °C, about 285 °C, about 290 °C, about 295 °C, about 300 °C, about 305 °C, about 310 °C, about 315 °C, about 320 °C, about 325 °C, about 350 °C, about 400 °C, or within any range encompassed by any two of the foregoing values as endpoints. For example, the reaction temperature is from about 100 °C to about 400 °C, or from about 125 °C to about 350 °C. The temperature is preferably from about 100 °C to about 350 °C, and more preferably from about 200 °C to about 300 °C.

### Process 1 Reaction (ii) Contact Time

For reactions using each catalyst/support combination (each row) in Table 2, the contact time of the reactants with the catalyst (Pd, Pt, Rh) in Reaction (ii) of Process 1 is as little as about 0.1 second, about 1 second, about 2 seconds, about 5 seconds, about 10 seconds, about 15 seconds, about 20 seconds, or as long as about 25 seconds, about 30 seconds, about 40 seconds, about 50 seconds, about 60 seconds, about 80 seconds, about 120 seconds, or within any range encompassed by two of the foregoing values as endpoints, for example from about 0.1 seconds to about 120 seconds, from about 1 second to about 60 seconds, from about 5 seconds to about 50 seconds, from about 10 seconds to about 40 seconds, from about 15 seconds to about 30 seconds, or about 20 seconds to about 25 seconds. For example, the contact time is from about 1 second to about 60 seconds.

### Process 1 Reaction (ii) Pressure

For reactions using each catalyst/support combination (each row) in Table 2, the pressure inside which reactor Reaction (ii) of Process 1 takes place is as little as about 1 psig, about 3 psig, about 5 psig, about 10 psig, about 15 psig, about 20 psig, about 30 psig, about 35 psig or about 40 psig, or as great as about 90 psig, about 100 psig, about 120 psig, about 150 psig, about 200 psig or about 250 psig, about 300 psig, or within any range encompassed by two of the foregoing values as endpoints, for example from about 1 psig to about 300 psig, from about 3 psig to about 250 psig, from about 5 psig to about 200 psig, from about 10 psig to about 150 psig, from about 15 psig to about 120 psig, from about 20 psig to about 100 psig, from about 30 psig to about 90 psig, or from about 35 psig to about 40 psig. For example, the pressure is from about 10 psig to about 200 psig.

### Process 1 Reaction (ii) Hydrogen Mole Ratio

For reactions using each catalyst/support combination (each row) in Table 2 used in Reaction (ii) of Process 1, the mole ratio of hydrogen to CFC-113 is as little about 2:1, about 3:1, about 4:1, about 5:1, about 5.5:1 or as great as about 6:1, about 6.5:1, about 7.5:1 or about 8:1, about 12:1, about 15:1, or about 20:1, for example, or within any range encompassed by two of the foregoing values as endpoints. The mole ratio of hydrogen to CFC-113 is preferably from about 3:1 about to 15:1, and more preferably from about 4:1 to about 10:1.

### Process 1 Reaction (ii) Products—Selectivity to HFC-143

As demonstrated by the Examples herein, for reactions using each catalyst/support combination (each row) in Table 2, Reaction (ii) of Process 1 may achieve a selectivity to the desired product 1,1,2-trifluoroethane (HFC-143) of at least about 20%, at least about 30%, at least about 40%, at least about 50%, at least about 60%, at least about 70%, at least about 80%, at least about 90%, and for each of the foregoing, less than or equal to 100%, or within any range encompassed by two of the foregoing values as endpoints, for example from about 20% to about 90%, from about 30% to about 80%, from about 40% to about 80%, or about 50% to about 80%, based on total moles of the organic components of the composition.

In one example, Reaction (ii) of Process 1 is carried out in the reactor 120 using palladium metal catalyst on a catalyst support (Pd/C) with the Pd loading from about 1 wt. % to about 5 wt. % palladium metal based on a combined weight of the palladium metal and the carbon support, at a temperature from about 200 °C to about 350 °C.

In another example, Reaction (ii) of Process 1 is carried out in the reactor 106 using palladium metal catalyst (Pd) on a catalyst support (Activated Carbon) with the Pd loading from about 2 wt. % to about 4 wt. % palladium metal based on a combined weight of the palladium metal and the carbon support, at a temperature from about 200 °C to about 325 °C.

In another example, Reaction (ii) of Process 1 is carried out in the reactor 106 using palladium metal catalyst on a catalyst support (Pd/C) with the Pd loading at about 4 wt. % palladium metal based on a combined weight of the palladium metal and the carbon support, at a temperature from about 250 °C to about 350 °C.

In one example, Reaction (ii) of Process 1 is carried out in the reactor 120 using platinum metal catalyst on a catalyst support (Pt/C) with the Pt loading from about 1 wt. % to about 5 wt. % platinum metal based on a combined weight of the platinum metal and the carbon support, at a temperature from about 200 °C to about 350 °C.

In another example, Reaction (ii) of Process 1 is carried out in the reactor 120 using platinum metal catalyst (Pt) on a catalyst support (Activated Carbon) with the Pt loading from about 2 wt. % to about 4 wt. % platinum metal based on a combined weight of the platinum metal and the carbon support, at a temperature from about 200 °C to about 325 °C.

In another example, Reaction (ii) of Process 1 is carried out in the reactor 120 using platinum metal catalyst on a catalyst support (Pt/C) with the Pt loading at about 4 wt. % platinum metal based on a combined weight of the platinum metal and the carbon support, at a temperature from about 250 °C to about 350 °C.

In one example, Reaction (ii) of Process 1 is carried out in the reactor 120 using rhodium metal catalyst on a catalyst support (Rh/C) with the Rh loading from about 1 wt. % to about 5 wt. % rhodium metal based on a combined weight of the rhodium metal and the carbon support, at a temperature from about 200 °C to about 350 °C.

In another example, Reaction (ii) of Process 1 is carried out in the reactor 120 using rhodium metal catalyst (Rh) on a catalyst support (Activated Carbon) with the Rh loading from about 2 wt. % to about 4 wt. % rhodium metal based on a combined weight of the rhodium metal and the carbon support, at a temperature from about 200 °C to about 325 °C.

In another example, Reaction (ii) of Process 1 is carried out in the reactor 120 using rhodium metal catalyst on a catalyst support (Rh/C) with the Rh loading at about 4 wt. % rhodium metal based on a combined weight of the rhodium metal and the carbon support, at a temperature from about 250 °C to about 350 °C.

Process 1 Reaction (ii)—Conversion of CFC-113

For Reaction (ii) of Process 1 carried out using each catalyst/support combination (each row) in Table 2, the reaction achieves a conversion of at least about 50%, at least about 60%, at least about 70%, at least about 80%, or at least about 90%, and for each of the foregoing, less than or equal to 100%, or within any range encompassed by two of the foregoing values as endpoints, for example from about 50% to about 100%, or from about 60% to about 100%, based on total moles of the organic components of the composition.

Process 1 Reaction (ii)—Product Stream from Reactor

Referring again to FIG. 1, Reaction (ii) of Process 1 inside reactor 120 produces a product stream 122 (before any post processing steps) comprising the target product 1,1,2-trifluoroethane (HFC-143), recyclable intermediates including but not limited to 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a), unreacted starting materials including but not limited to hydrogen (H₂) and 1,1,2-trichloro-1,2,2-trifluoroethane (CFC-113), and other byproducts including but not limited to ethane (HC-170), 1,1-difluoroethane (HFC-152a), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), chloroethane (HCC-160), 1,1,1-trifluoroethane (HFC-143a), hydrogen chloride (HCl), and hydrogen fluoride (HF).

For Reaction (ii) of Process 1 carried out using each catalyst/support combination (each row) in Table 2, referring to FIG. 1, the amount of unreacted CFC-113 in the product composition in stream 122 from the reactor 120, if present, is greater than or equal to 0.1 mol % and yet less than 2 mol %, less than 1 mol %, or less than 0.5 mol %, for example, based on total moles of organic components of the composition.

For Reaction (ii) of Process 1 carried out using each catalyst/support combination (each row) in Table 2, referring to FIG. 1, the amount of 1,1,2-trifluoroethane (HFC-143) in the product composition in stream 122 from the reactor 120 is at least 40 mol %, at least 60 mol %, or at least 70 mol %, and for each of the foregoing, less than or equal to 100 mol %, for example, based on total moles of organic components of the composition.

For Reaction (ii) of Process 1 carried out using each catalyst/support combination (each row) in Table 2, referring to FIG. 1, the amount of 1-chloro-1,1,2-trifluoroethane (HCFC-133b) in the product composition in stream 122 from the reactor 120, if present, is greater than or equal to 0.5% and yet less than 40 mol %, less than 30 mol %, less than 25 mol %, or less than 20 mol %, for example, based on total moles of organic components of the composition.

For Reaction (ii) of Process 1 carried out using each catalyst/support combination (each row) in Table 2, referring to FIG. 1, the amount of 1-chloro-1,2,2-trifluoroethane (HCFC-133) in the product composition in stream 122 from the reactor 120, if present, is greater than or equal to 0.1 mol % and yet less than 10 mol %, less than 5 mol %, or less than 1 mol %, for example, based on total moles of organic components of the composition.

For Reaction (ii) of Process 1 carried out using each catalyst/support combination (each row) in Table 2, referring to FIG. 1, the amount of 1,2-dichloro-1,1,2-trifluoroethane (HCFC-123a) in the product composition in stream 122 from the reactor 120, if present, is greater than or equal to 0.1 mol % and yet less than 15 mol %, less than 5 mol %, or less than 0.5 mol %, for example, based on total moles of organic components of the composition.

For Reaction (ii) of Process 1 carried out using each catalyst/support combination (each row) in Table 2, referring to FIG. 1, the total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) in the product composition in stream 122 from the reactor 120 is at least 80 mol %, at least 85 mol %, or at least 90 mol %, and for each of the foregoing, less than or equal to 100 mol %, for example, based on total moles of organic components of the composition.

For example, for Reaction (ii) of Process 1 carried out using each catalyst/support combination (each row) in Table 2, referring to FIG. 1, in the product composition in stream 122 from the reactor 120, the amount of 1,1,2-trifluoroethane (HFC-143) is at least 45 mol % and less than or equal to 99.3 mol %, the amount of 1-chloro-1,1,2-trifluoroethane (HCFC-133b) is at least 0.5 mol % and less than or equal to 40 mol %, the amount of 1-chloro-1,2,2-trifluoroethane (HCFC-133) is at least 0.1 mol % and less than or equal to 5 mol %, and the amount of 1,2-dichloro-1,1,2-trifluoroethane (HCFC-123a) is at least 0.1 mol % and less than or equal to 10 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph is at least 80 mol % and less than or equal to 100% of the total moles of organic components of the product composition in stream 122 from the reactor 120, while the combined amount of other components including undesired byproducts (e.g., HFC-143a, HC-170, HCC-160, HFC-152a, and HCFC-142 isomers) is greater than or equal to 0 mol % and less than 20 mol % of the total moles of organic components of the product composition in stream 122 from the reactor 120.

For example, for Reaction (ii) of Process 1 carried out using each catalyst/support combination (each row) in Table 2, referring to FIG. 1, in the product composition in stream 122 from the reactor 120, the amount of 1,1,2-trifluoroethane (HFC-143) is at least 65 mol % and less than or equal to 99.3 mol %, the amount of 1-chloro-1,1,2-trifluoroethane (HCFC-133b) is at least 0.5 mol % and less than or equal to 25 mol %, the amount of 1-chloro-1,2,2-trifluoroethane (HCFC-133) is at least 0.1 mol % and less than or equal to 5 mol %, and the amount of 1,2-dichloro-1,1,2-trifluoroethane (HCFC-123a) is at least 0.1 mol % and less than or equal to 5 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph is at least 85 mol % and less than or equal to 100% of the total moles of organic components of the product composition in stream 122 from the reactor 120, while the combined amount of other components including undesired byproducts (e.g., HFC-143a, HC-170, HCC-160, HFC-152a, and HCFC-142 isomers) is greater than or equal to 0 mol % and less than 15 mol % of the total moles of organic components of the product composition in stream 122 from the reactor 120.

For example, for Reaction (ii) of Process 1 carried out using each catalyst/support combination (each row) in Table 2, referring to FIG. 1, in the product composition in stream 122 from the reactor 120, the amount of 1,1,2-trifluoroethane (HFC-143) is at least 78.5 mol % and less than or equal to 99.3 mol %, the amount of 1-chloro-1,1,2-trifluoroethane (HCFC-133b) is at least 0.5 mol % and less than or equal to 20 mol %, the amount of 1-chloro-1,2,2-trifluoroethane (HCFC-133) is at least 0.1 mol % and less than or equal to 1 mol %, and the amount of 1,2-dichloro-1,1,2-trifluoroethane (HCFC-123a) is at least 0.1 mol % and less than or equal to 0.5 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph is at least 90 mol % and less than or equal to 100% of the total moles of organic components of the product composition in stream 122 from the reactor 120, while the combined amount of other components including undesired byproducts (e.g., HFC-143a, HC-170, HCC-160, HFC-152a, and HCFC-142 isomers) is greater than or equal to 0 mol % and less than 10 mol % of the total moles of organic components of the product composition in stream 122 from the reactor 120.

Process 1 Reaction (ii)—Product Stream Composition Post Processing

Referring again to FIG. 1, separation unit 124 may include one or more distillation columns to remove undesired byproducts of the hydrogenation reaction and other impurities from product stream 122. For example, the separation unit 124 may include a first distillation column to remove unreacted hydrogen (H₂), a second distillation column to remove hydrogen chloride (HCl) and ethane (HC-170), a third distillation column to remove low boiling point compounds including but not limited to 1,1-difluoroethane (HFC-152a), 1-chloro-1,2-difluoroethane (HCFC-142b), trifluoroethylene (HFO-1123), and 1,1,1-trifluoroethane (HFC-143a), and a fourth distillation column to further purify the product mixture and produce product stream 126 including predominantly HFC-143. Each distillation column is operated at pressures ranging from 0 to 300 psig. Temperatures of the distillation columns will be determined by the selected pressure and composition of the overhead and bottoms of the distillation columns. Separation unit 124 may also include one or more scrubbers for removing hydrogen fluoride (HF) and hydrogen chloride (HCl) from the product mixture. Other unit operations may also be employed alone or in combination with distillation columns. Such unit operations may include one or more of, or a combination of, columns (e.g., distillation columns), scrubbers, adsorbers, absorbers, membrane separation units, flash vessels, vaporizers, etc.

For Reaction (ii) of Process 1 carried out using each catalyst/support combination (each row) in Table 2, the amount or purity of 1,1,2-trifluoroethane (HFC-143) in overhead stream 126 from separation unit 124 is at least 88 mol %, at least 89 mol %, at least 90 mol %, or at least 91 mol %, and for each of the foregoing, less than or equal to 100 mol %, for example, based on total moles of organic components in the composition.

For Reaction (ii) of Process 1 carried out using each catalyst/support combination (each row) in Table 2, the amount of 1-chloro-1,1,2-trifluoroethane (HCFC-133b) in overhead stream 126 is less than 2000 ppm, less than 1000 ppm, less than 500 ppm, or less than 250 ppm, for example, based on total moles of organic components in the composition.

Process 1 Reaction (ii)—Recycle Stream Composition

For Reaction (ii) of Process 1 carried out using each catalyst/support combination (each row) in Table 2, a bottom recycle stream 128 from separation unit 124 comprising desired intermediates including but not limited to 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a) may be recycled back into reactor 120 so that the foregoing molecules may be further reacted in a continuous manner according to the present single reactor method. Optionally, bottom recycle stream 128 may be sent to a second reactor (not shown).

For Reaction (ii) of Process 1 carried out using each catalyst/support combination (each row) in Table 2, referring to FIG. 1, the amount of 1,1,2-trifluoroethane (HFC-143) in the recycle stream 128, if present, is less than 50 mol %, less than 40 mol %, or less than 3 mol %, and for each of the foregoing, greater than or equal to 0.01 mol %, for example, based on total moles of organic components of the composition.

For Reaction (ii) of Process 1 carried out using each catalyst/support combination (each row) in Table 2, referring to FIG. 1, the amount of 1-chloro-1,1,2-trifluoroethane (HCFC-133b) in the recycle stream 128 is greater than 5 mol %, greater than 10 mol %, greater than 15 mol %, greater than 20 mol %, greater than 40 mol %, or greater than 60 mol %, and for each of the foregoing, less than or equal to 100 mol %, for example, based on total moles of organic components of the composition.

For Reaction (ii) of Process 1 carried out using each catalyst/support combination (each row) in Table 2, referring to FIG. 1, the amount of 1-chloro-1,2,2-trifluoroethane (HCFC-133) in the recycle stream 128, if present, is less than 10 mol %, less than 5 mol %, or less than 1 mol %, and for each of the foregoing, greater than or equal to 0.01 mol %, for example, based on total moles of organic components of the composition.

For Reaction (ii) of Process 1 carried out using each catalyst/support combination (each row) in Table 2, referring to FIG. 1, the amount of 1,2-dichloro-1,1,2-trifluoroethane (HCFC-123a) in the recycle stream 128, if present, is less than 20 mol %, less than 15 mol %, or less than 9 mol %, and for each of the foregoing, greater than or equal to 0.01 mol %, for example, based on total moles of organic components of the composition.

For Reaction (ii) of Process 1 carried out using each catalyst/support combination (each row) in Table 2, referring to FIG. 1, the total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) in the recycle stream 128 is at least 80 mol %, at least 90 mol %, or at least 95 mol %, and for each of the foregoing, less than or equal to 100 mol %, for example, based on total moles of organic components of the composition.

For example, for Reaction (ii) of Process 1 carried out using each catalyst/support combination (each row) in Table 2, referring to FIG. 1, in the recycle stream 128, the amount of 1,1,2-trifluoroethane (HFC-143) is less than 50 mol % and greater than or equal to 0 mol %, the amount of 1-chloro-1,1,2-trifluoroethane (HCFC-133b) is at least 20 mol % and less than or equal to 100 mol %, the amount of 1-chloro-1,2,2-trifluoroethane (HCFC-133) is less than 10 mol % and greater than or equal to 0 mol %, the amount of 1,2-dichloro-1,1,2-trifluoroethane (HCFC-123a) is less than 20 mol % and greater than or equal to 0 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph is at least 80 mol % and less than or equal to 100% of the total moles of organic components of the product composition in the recycle stream 128, while the combined amount of other components including undesired byproducts (e.g., HFC-143a, HC-170, HCC-160, HFC-152a, and HCFC-142 isomers) is greater than or equal to 0 mol % and less than 20 mol % of the total moles of organic components of the product composition in the recycle stream 138 to the reactor 106.

For example, for Reaction (ii) of Process 1 carried out using each catalyst/support combination (each row) in Table 2, referring to FIG. 1, in the recycle stream 128, the amount of 1,1,2-trifluoroethane (HFC-143) is less than 40 mol % and greater than or equal to 0 mol %, the amount of 1-chloro-1,1,2-trifluoroethane (HCFC-133b) is greater than 40 mol % and less than or equal to 100 mol %, the amount of 1-chloro-1,2,2-trifluoroethane (HCFC-133) is less than 5 mol % and greater than or equal to 0 mol %, the amount of 1,2-dichloro-1,1,2-trifluoroethane (HCFC-123a) is less than 15 mol % and greater than or equal to 0 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph is at least 90 mol % and less than or equal to 100% of the total moles of organic components of the product composition in the recycle stream 128, while the combined amount of other components including undesired byproducts (e.g., HFC-143a, HC-170, HCC-160, HFC-152a, and HCFC-142 isomers) is greater than or equal to 0 mol % and less than 10 mol % of the total moles of organic components of the product composition in the recycle stream 138 to the reactor 106.

For example, Reaction (ii) of Process 1 carried out using each catalyst/support combination (each row) in Table 2, referring to FIG. 1, in the recycle stream 128, the amount of 1,1,2-trifluoroethane (HFC-143) is less than 30 mol % and greater than or equal to 0 mol %, the amount of 1-chloro-1,1,2-trifluoroethane (HCFC-133b) is greater than 60 mol % and less than or equal to 100 mol %, the amount of 1-chloro-1,2,2-trifluoroethane (HCFC-133) is less than 1 mol % and greater than or equal to 0 mol %, the amount of 1,2-dichloro-1,1,2-trifluoroethane (HCFC-123a) is less than 9 mol % and greater than or equal to 0 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph is at least 95 mol % and less than or equal to 100% of the total moles of organic components of the product composition in the recycle stream 128, while the combined amount of other components including undesired byproducts (e.g., HFC-143a, HC-170, HCC-160, HFC-152a, and HCFC-142 isomers) is greater than or equal to 0 mol % and less than 5 mol % of the total moles of organic components of the product composition in the recycle stream 138 to the reactor 106.

### IV. Process 2

### Process Flow

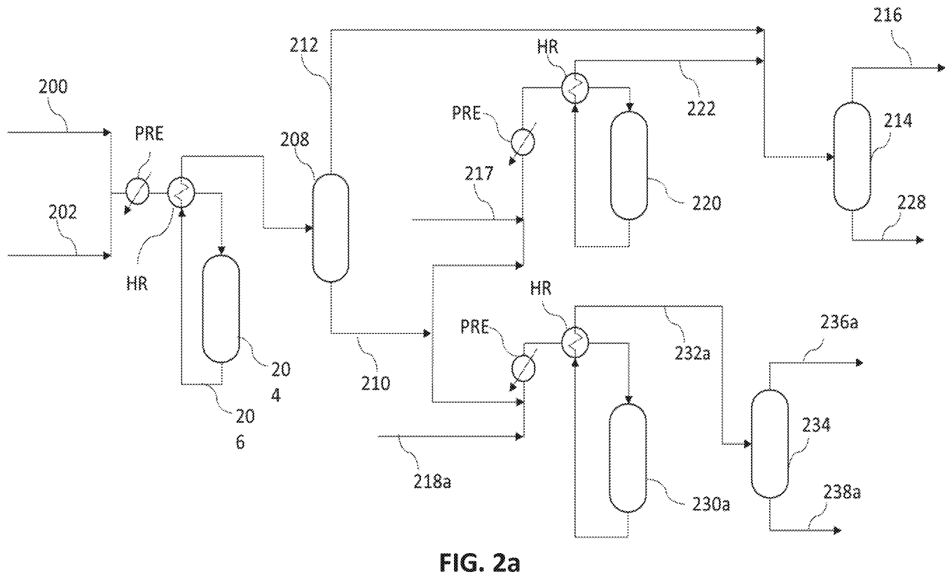

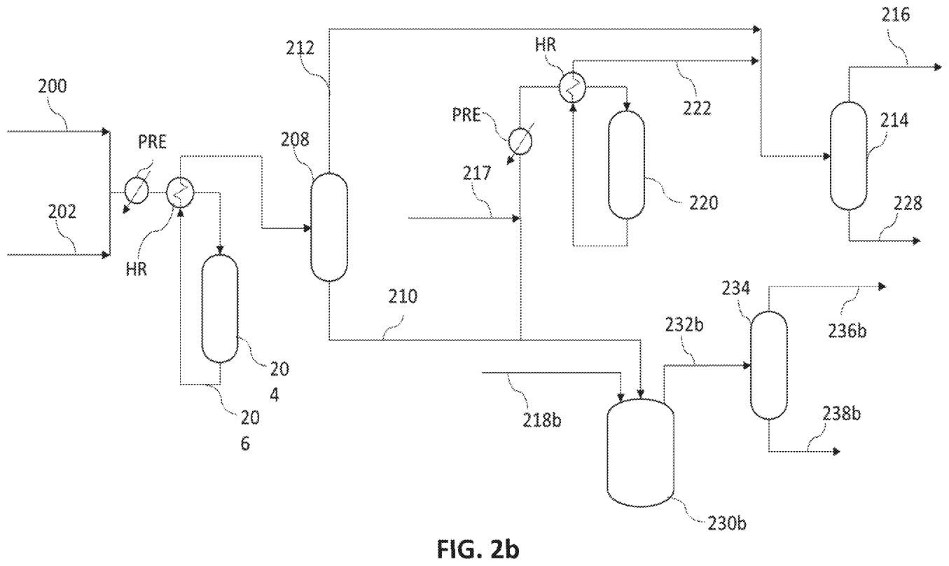

FIGS. 2a and 2b include schematic process flow diagrams illustrating suitable components for the reactions in Process 2 converting CFCl₂—CF₂Cl (CFC-113) to CF₂═CFCl (CTFE) and/or CFH₂—CF₂H (HFC-143).

Referring to FIGS. 2a and 2b, stream 200 comprising hydrogen (H₂) and stream 202 comprising CFC-113 enter reactor 204. Inside reactor 204, the CFC-113 undergoes the hydrogenation reaction, Step (i) of Process 2, to produce a first product composition comprising HCFC-123a. Stream 206 including first product composition enters separation unit 208 for separating HCFC-123a from HFC-143 and other byproducts (e.g., trifluoroethene, 1-chloro-1,2,2-trifluoroethane (HCFC-133), hydrogen chloride (HCl), etc.) and unreacted reactants (e.g., hydrogen and CFC-113). The separation unit 208 may include one or more of, or a combination of, columns (e.g., distillation columns), scrubbers, adsorbers, absorbers, membrane separation units, flash vessels, vaporizers, etc.

Top stream 212 from the separation unit 208 containing HFC-143 and other byproducts enters separation unit 214 to produce desired product HFC-143 in stream 216. Bottom stream 210 from the separation unit 208 containing predominantly HCFC-123a is used as feed for either Step (ii) hydrogenation reaction of Process 2—Pathway I to produce HFC-143 in reactor 220, or Step (ii) dehydrochlorination reaction of Process 2—Pathway II to produce CTFE in vapor phase reactor 230a or liquid phase reactor 230b.

The flow of stream 210 (whether it enters reactor 220 or 230a-b) is controlled depending on the desired end product. The control may be, for example, by one or more selective valving arrangements, one or more feed storage tanks, etc. For example, when the desired end product is CTFE, 100% of stream 210 is configured to enter reactor 230a or reactor 230b. When the desired end product is HFC-143, 100% of stream 210 is configured to enter reactor 220. When the desired end products are both CTFE and HFC-143, stream 210 is configured to split the feed based on desired product ratio to enter a fraction (e.g., 0% to 100%) to reactor 220 and the remaining fraction (e.g., 100% to 0%) to enter reactor 230a-b. Additional feed including hydrogen may be supplied through streams 217 and 218 to vapor phase reactors 220 and 230a to adjust the amount and ratio of reactant composition that enters the reactors. For liquid phase reactor 230b, stream 218b including a base is supplied for carrying out the dehydrochlorination reaction, Step (ii) of Process 2—Pathway II, under liquid phase.

Inside reactor 220, where the reaction system is Process 2—Pathway I, the HCFC-123a undergoes a Step (ii) hydrogenation reaction to produce a second product composition comprising HFC-143. Stream 222 including second product composition enters separation unit 214 (e.g., columns, scrubbers, adsorbers, absorbers, membrane separation units, flash vessels, vaporizers, etc.) for producing stream 216 including predominantly HFC-143 and recycle stream 228.

Inside reactor 230a, referring to FIG. 2a, where the reaction system is Process 2—Pathway II, the HCFC-123a undergoes the Step (ii) Vapor Phase dehydrochlorination reaction to produce a second product composition comprising CTFE. Stream 232a including a second product composition enters separation unit 234 (e.g., columns, scrubbers, adsorbers, absorbers, membrane separation units, flash vessels, vaporizers, etc.) for producing stream 236a including predominantly CTFE and recycle stream 238a.

Inside reactor 230b, referring to FIG. 2b, where the reaction system is Process 2—Pathway II, the HCFC-123a undergoes the Step (ii) Liquid Phase dehydrochlorination reaction to produce a second product composition comprising CTFE. Stream 232b including a second product composition enters separation unit 234 (e.g., columns, scrubbers, adsorbers, absorbers, membrane separation units, flash vessels, vaporizers, etc.) for producing stream 236b including predominantly CTFE and recycle stream 238b.

The hydrogenation reactions and vapor phase dehydrochlorination reactions of Step (i) and Step (ii) of Process 2 may be carried out in the gas or vapor phase in a suitable reactor, for example a tubular reactor made from a material which is resistant to temperature and/or corrosion including but not limited to nickel and its alloys, including Hastelloy (for example, Hastelloy C276), Inconel (for example, Inconel 600), Incoloy (for example, Incoloy 825), and Monel (for example, Monel 400), and the vessels may be lined with fluoropolymers.

The reactors (e.g., reactor 204, reactor 220, reactor 230a) used for the vapor phase reactions of Step (i) and Step (ii) of Process 2 may be first cleaned and flushed with an inert gas including but not limited to nitrogen, followed by packing with a catalyst including but not limited to those described below. The catalyst may be pretreated within the reactor including but not limited to by drying in the manner described further below, followed by metering the reactants into the reactor to initiate the reaction.

The process flow for each of the Reaction (i) or Reaction (ii) may be in the down or up direction through a bed of the catalyst. Products may be flowed through one or more separation units (e.g., separation unit 208, 214, 234a-b) to remove undesired byproducts from the reaction, including but not limited to hydrogen fluoride (HF) and/or hydrogen chloride (HCl), and the reaction products may be collected in a suitable vessel, for example.

As shown throughout FIGS. 2a and 2b, each vapor phase reactor (e.g., reactor 204, reactor 220, reactor 230a) may optionally be equipped with a pre-heater to pre-heat the reactants prior to entering the reactor. In addition, each vapor phase reactor (e.g., reactor 204, reactor 220, reactor 230a) may optionally be equipped with a heat recovery unit to cool down hot reactor effluent by heating the incoming reactor feed.

The catalysts and process conditions for each of Step (i) and Step (ii) in Process 2 under different pathways (e.g., Pathway I for producing HFC-143, Pathway II for producing CTFE)) are discussed in more detail below.

### Process 2—Pathway I & Pathway II Step (i) Catalysts

In the hydrogenation reaction of Process 2 Step (i), for both Pathway I and Pathway II, the catalyst may comprise a metal including but not limited to palladium, platinum, rhodium, ruthenium, iron, cobalt or nickel. The catalyst active to catalyze the reaction may preferably be palladium metal (Pd), platinum metal (Pt), or a combination of palladium metal and platinum metal.

### Process 2—Pathway I & Pathway II Step (i) Catalyst Support

In the hydrogenation reaction of Process 2 Step (i), for both Pathway I and Pathway II, the catalyst may be supported on a suitable support, including but not limited to carbon or alumina (aluminum oxide—Al₂O₃). The carbon may be activated carbon. The alumina may be alpha(α) alumina, theta(θ) alumina, delta(δ) alumina, or gamma(γ) alumina. The supported catalyst may be produced by impregnation of any of the suitable supports with a solution of a compound of the desired metal constituent. The support may also be in the form of pellets. After the impregnation step, the solvent may be removed using heat or under vacuum resulting in a solid mass which can be further dried and reduced to form active metal catalyst.

In the hydrogenation reaction of Process 2 Step (i), for both Pathway I and Pathway II, the catalyst may be palladium on a carbon support, may be platinum on a carbon support, and/or may be palladium or platinum on an alumina support. The catalyst may be palladium on a carbon support or palladium on an alpha alumina support.

In the hydrogenation reaction of Process 2 Step (i), for both Pathway I and Pathway II, the metal catalysts supported on various catalyst supports used are listed in Table 3 below.

TABLE 3
Supported Metal Catalysts - Step (i) Hydrogenation Reaction

| Catalyst | Support |
|---|---|
| Pd | Activated Carbon |
| Pd | alpha(α)-Al₂O₃ |
| Pd | theta(θ)-Al₂O₃ |
| Pd | delta(δ)-Al₂O₃ |
| Pd | gamma(γ)-Al₂O₃ |
| Pt | Activated Carbon |
| Pt | alpha(α)-Al₂O₃ |
| Pt | theta(θ)-Al₂O₃ |
| Pt | delta(δ)-Al₂O₃ |
| Pt | gamma(γ)-Al₂O₃ |

### Process 2—Pathway I & Pathway II Step (i) Catalyst Loading

For each catalyst/support combination (each row) in Table 3 used in the hydrogenation reaction of Step (i) of Process 2, for both Pathway I and Pathway II, the amount of metal loading on the support is from about 0.01 wt. %, about 0.05 wt. %, about 0.1 wt. %, about 0.2 wt. %, about 0.3 wt. %, about 0.4 wt. %, about 0.5 wt. %, or about 1 wt. % to about 2 wt. %, about 3 wt. %, about 4 wt. %, about 5 wt. %, about 10 wt. %, about 20 wt. %, about 30 wt. %, about 40 wt. %, about 50 wt. % or within any range encompassed by two of the foregoing values as endpoints, for example, from about 0.01 wt. % to about 20 wt. %, from about 0.01 wt. % to about 10 wt. %, from about 0.1 wt. % to about 10 wt. %, 0.2 wt. % to about 10 wt. %, from about 0.5 wt. % to about 5 wt. %, from about 1 wt. % to about 5 wt. %, from about 1 wt. % to about 4 wt. %, or about 2 wt. % to about 4 wt. % based on a total weight of the catalyst and support. For supported noble metal catalysts including but not limited to Pd or Pt, the metal loading is from about 0.01 wt. % to about 5 wt. % based on a total weight of the catalyst and support.

### Process 2—Pathway I & Pathway II Step (i) Catalyst BET Surface Area

For each catalyst/support combination (each row) in Table 3 used in the hydrogenation reaction of Step (i) of Process 2, for both Pathway I and Pathway II, the BET surface area of the catalyst may be as low as about 0.5 m²/g, about 1 m²/g, about 3 m²/g, about 5 m²/g, about 10 m²/g, about 15 m²/g, about 20 m²/g², about 30 m²/g, about 40 m²/g, about 50 m²/g, about 100 m²/g, about 200 m²/g, or as high as about 250 m²/g, about 300 m²/g, about 400 m²/g, about 500 m²/g, about 600 m²/g, about 700 m²/g m², about 800 m²/g, about 900 m²/g, about 1000 m²/g, about 2000 m²/g, about 3000 m²/g, or within any range encompassed by any of the foregoing values as endpoints, for example from about 0.5 m²/g to about 3000 m²/g, from about 1 m²/g to about 2000 m²/g, from about 1000 m²/g to about 2000 m²/g, from about 0.5 m²/g to about 500 m²/g, from about 0.5 m²/g to about 300 m²/g, from about 0.5 m²/g to about 5 m²/g, or from about 200 m²/g to about 300 m²/g.

### Process 2—Pathway I & Pathway II Step (i) Catalyst Pretreatment

For each catalyst/support combination (each row) in Table 3 used in the hydrogenation reaction of Step (i) of Process 2, for both Pathway I and Pathway II, the catalyst may be pretreated by a variety of methods to improve its performance and effectiveness in the reaction. For example, the catalyst may be dried at elevated temperatures, as low as about 200 °C, about 250 °C, about 300 °C, about 350 °C, about 360 °C, about 370 °C, or as high as about 380 °C, about 390 °C, about 400 °C, about 450 °C, about 500 °C, about 600 °C, about 700 °C, or within any range encompassed by two of the foregoing values as endpoints, for example from about 200 °C to about 400 °C, from about 200 °C to about 350 °C, from about 250 °C to about 350 °C, from about 250 °C to about 300 °C, or from about 260 °C to about 300 °C.

### Process 2—Pathway I & Pathway II Step (i) Reaction Temperature

For reactions using each catalyst/support combination (each row) in Table 3, the reaction temperature of the hydrogenation reaction of Step (i) of Process 2, for both Pathway I and Pathway II, is as low as about 100 °C, about 125 °C, about 150 °C, about 200 °C, about 230 °C, about 250 °C or as high as about 300 °C, about 350 °C, about 400 °C, or within any range encompassed by two of the foregoing values as endpoints, for example from about 100 °C to about 400 °C, or from about 125 °C to about 350 °C, from about 150 °C to about 300 °C, or from about 200 °C to about 250 °C, for example. The temperature is preferably from about 100 °C to about 350 °C, and more preferably from about 200 °C to about 300 °C.

### Process 2—Pathway I & Pathway II Step (i) Reaction Contact Time

For reactions using each catalyst/support combination (each row) in Table 3, the contact time of the reactants with the catalyst (Pd and Pt) in the hydrogenation reaction of Step (i) of Process 2, for both Pathway I and Pathway II, is as little as about 0.1 second, about 1 second, about 2 seconds, about 5 seconds, about 10 seconds, about 15 seconds or about 20 seconds, or as long as about 25 seconds, about 30 seconds, about 40 seconds, about 50 seconds, about 60 seconds, about 120 seconds, or within any range encompassed by two of the foregoing values as endpoints, for example from about 0.1 seconds to about 120 seconds, from about 1 second to about 60 seconds, from about 5 seconds to about 50 seconds, from about 10 seconds to about 40 seconds, from about 15 seconds to about 30 seconds, or about 20 seconds to about 25 seconds. For example, the contact time is preferably from about 1 second to about 60 seconds, or more preferably from about 20 seconds to about 40 seconds.

### Process 2—Pathway I & Pathway II Step (i) Reaction Pressure

For reactions using each catalyst/support combination (each row) in Table 3, the pressure inside which reactor the hydrogenation reaction of Step (i) of Process 2, for both Pathway I and Pathway II, takes place is as little as about 1 psig, about 3 psig, about 5 psig, about 10 psig, about 15 psig, about 20 psig, about 30 psig, about 35 psig or about 40 psig, or as great as about 90 psig, about 100 psig, about 120 psig, about 150 psig, about 200 psig or about 250 psig, about 300 psig, or within any range encompassed by two of the foregoing values as endpoints, for example from about 1 psig to about 300 psig, from about 3 psig to about 250 psig, from about 5 psig to about 200 psig, from about 10 psig to about 150 psig, from about 15 psig to about 120 psig, from about 20 psig to about 100 psig, from about 30 psig to about 90 psig, or from about 35 psig to about 40 psig. For example, the pressure is preferably from about 10 psig to about 200 psig, or more preferably from about 50 psig to about 100 psig.

### Process 2—Pathway I & Pathway II Step (i) Reaction Hydrogen Molar Ratio

For reactions using each catalyst/support combination (each row) in Table 3 used in the hydrogenation reaction of Step (i) of Process 2, depending on the pathway (e.g., Pathway I of Process 2, Pathway II of Process 2) and corresponding desired end product (e.g., HFC-143, CTFE), the mole ratio of hydrogen to 1,1,2-trichloro-1,2,2-trifluoroethane (CFC-113) may be selectively tailored to drive the reaction towards a higher production of either HFC-123a or HFC-143 in the product composition.

For example, for reactions using each catalyst/support combination (each row) in Table 3 used in the hydrogenation reaction of Step (i) of Process 2, for Pathway 1, the mole ratio of hydrogen to CFC-113 is as little about 0.3:1, about 0.4:1, about 0.5:1, about 0.6:1, about 0.7:1, about 0.8:1, about 0.9:1, about 1:1, or as great as about 1.5:1, about 1.6:1, about 1.7:1, about 1.8:1, about 1.9:1, about 2:1, about 2.5:1, about 3:1, or within any range encompassed by two of the foregoing values as endpoints. For Step (i) of Process 2, Pathway 1, the mole ratio of hydrogen to CFC-113 is preferably from about 0.3:1 to about 3:1, and more preferably from about 0.5:1 to about 2:1.

For example, for reactions using each catalyst/support combination (each row) in Table 3 used in the hydrogenation reaction of Step (i) of Process 2, for Pathway II, the mole ratio of hydrogen to CFC-113 is as little about 1:1, about 1.5:1, about 2:1, about 2.5:1, about 3:1, about 3.5:1, about 4:1, or as great as about 5:1, about 5.5:1 about 6:1, about 7:1 or about 8:1, or within any range encompassed by two of the foregoing values as endpoints. For Step (i) of Process 2, Pathway II, the mole ratio of hydrogen to CFC-113 is from about 1:1 to about 8:1, and preferably from about 1.5:1 to about 4:1.

### Process 2—Pathway I & Pathway II Step (i) Reaction—Conversion of CFC-113

As demonstrated by the Examples herein, for reactions using each catalyst/support combination (each row) in Table 3 used in the hydrogenation reaction of Step (i) of Process 2, for both Pathways I and II, the reaction may achieve a conversion of at least about 25%, at least about 30%, at least about 40%, at least about 50%, at least about 60%, at least about 70%, or at least about 80%, and for each of the foregoing, less than or equal to 100%, or within any range encompassed by two of the foregoing values as endpoints, for example from about 50% to about 100%, or from about 60% to about 100%, based on total moles of the organic components of the composition.

### Process 2—Pathway I & Pathway II Step (i) Reaction Products—Selectivity to HFC-123a and HFC-143

As demonstrated by the Examples herein, for reactions using each catalyst/support combination (each row) in Table 3 used in the hydrogenation reaction of Step (i) of Process 2, for both Pathways I and II, the reaction may achieve a selectivity to the desired product HFC-143 of at least about 10%, at least about 20%, at least about 30%, at least about 40%, at least about 50%, at least about 60%, at least about 70%, at least about 80%, at least about 90%, and for each of the foregoing, less than or equal to 100%, or within any range encompassed by two of the foregoing values as endpoints, for example from about 20% to about 90%, from about 20% to about 80%, from about 20% to about 70%, or about 20% to about 60%, based on total moles of the organic components of the composition.

As demonstrated by the Examples herein, for reactions using each catalyst/support combination (each row) in Table 3 used in the hydrogenation reaction of Step (i) of Process 2, for both Pathways I and II, the reaction may achieve a selectivity to the desired product HFC-123a of at least about 20%, at least about 30%, at least about 40%, at least about 50%, at least about 60%, at least about 70%, at least about 80%, at least about 90%, at least about 92%, at least about 93%, and for each of the foregoing, less than or equal to 100%, or within any range encompassed by two of the foregoing values as endpoints, for example from about 20% to about 100%, from about 40% to about 99%, from about 60% to about 98%, or about 80% to about 97%, based on total moles of the organic components of the composition.

Process 2—Pathway I & Pathway II Step (i)—Product stream from reactor

Referring to FIGS. 2a-2b, Step (i) of Process 2, for both Pathway I and II, the reaction inside reactor 204 produces a product stream 206 (before any post processing steps) comprising target products HFC-143, HFC-123a, and CTFE depending on the desired end product, recyclable intermediates including but not limited to 1-chloro-1,1,2-trifluoroethane (HCFC-133b) and/or 1-chloro-1,2,2-trifluoroethane (HCFC-133), unreacted starting materials including but not limited to hydrogen (H₂) and 1,1,2-trichloro-1,2,2-trifluoroethane (CFC-113), and other undesired byproducts including but not limited to ethane (HC-170), 1,1-difluoroethane (HFC-152a), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), chloroethane (HCC-160), trifluoroethene (HFO-1123), 1,1,1-trifluoroethane (HFC-143a), hydrogen fluoride (HF), 1,1-difluoroethane (HFC-152), and fluoroethane (HFC-161).

For Step (i) of Process 2, Pathway I, carried out using each catalyst/support combination (each row) in Table 3, referring to FIGS. 2a-2b, the amount of unreacted CFC-113 in the product composition in stream 206 from the reactor 204 is less than 75 mol %, less than 70 mol %, less than 60 mol %, less than 50 mol %, less than 40 mol %, less than 30 mol %, less than 25 mol %, less than 20 mol %, less than 10 mol %, less than 5 mol %, less than 4 mol %, less than 3 mol %, less than 2 mol %, less than 1 mol %, and for each of the foregoing, greater than or equal to 0 mol %, for example, based on total moles of organic components of the composition.

For Step (i) of Process 2, Pathway I, carried out using each catalyst/support combination (each row) in Table 3, referring to FIGS. 2a-2b, the amount of HFC-143 in the product composition in stream 206 from the reactor 204 is at least 10 mol %, at least 20 mol %, at least 40 mol %, at least 50 mol %, or at least 60 mol %, and for each of the foregoing, less than or equal to 100 mol %, for example, based on total moles of organic components of the composition.

For Step (i) of Process 2, both Pathways I and II, carried out using each catalyst/support combination (each row) in Table 3, referring to FIGS. 2a-2b, the amount of HFC-123a in the product composition in stream 206 from the reactor 204 is at least 10 mol %, at least 20 mol %, at least 40 mol %, at least 50 mol %, at least 60 mol %, at least 70 mol %, at least 80 mol %, at least 90 mol %, or at least 93 mol %, and for each of the foregoing, less than or equal to 100 mol %, for example, based on total moles of organic components of the composition.

For Step (i) of Process 2, both Pathways I and II, carried out using each catalyst/support combination (each row) in Table 3, referring to FIGS. 2a-2b, the amount of CTFE in the product composition in stream 206 from the reactor 204 is at least 0.01 mol %, at least 0.1 mol %, at least 0.2 mol %, at least 0.5 mol %, at least 5 mol %, at least 6 mol %, at least 70 mol %, at least 8 mol %, or at least 9 mol %, and for each of the foregoing, less than or equal to 15 mol %, for example, based on total moles of organic components of the composition.

For Step (i) of Process 2, both Pathways I and II, carried out using each catalyst/support combination (each row) in Table 3, referring to FIGS. 2a-2b, the total amount of HFC-123a, HFC-143, and CTFE in the product composition in stream 206 from the reactor 204 is at least 60 mol %, at least 70 mol %, at least 80 mol %, at least 90 mol %, or at least 93 mol %, and for each of the foregoing, less than or equal to 100 mol %, for example, based on total moles of organic components of the composition.

For example, for Step (i) of Process 2, both Pathways I and II, carried out using each catalyst/support combination (each row) in Table 3, referring to FIGS. 2a-2b, in the product composition in stream 206 from the reactor 204, The amount of HFC-123a is at least 90 mol % and less than or equal to 95 mol %, and the amount of CTFE is at least 5 mol % and less than or equal to 10 mol %, based on the combined total moles of HFC-123a and CTFE in the product composition in stream 206 from the reactor 204.

The combined total moles of HFC-123a and CTFE in the composition set forth in the preceding paragraph is at least 50 mol % and less than or equal to 100% of the total moles of organic components of the product composition in stream 206 from the reactor 204, while the combined amount of other components including unreacted CFC-113 is greater than or equal to 0 mol % and less than 50 mol % of the total moles of organic components of the product composition in stream 206 from the reactor 204.

For example, for Step (i) of Process 2, both Pathways I and II, carried out using each catalyst/support combination (each row) in Table 3, referring to FIGS. 2a-2b, in the product composition in stream 206 from the reactor 204, The amount of HFC-123a is at least 60 mol % and less than or equal to 70 mol %, the amount of HFC-143 is at least 7.5 mol % and less than or equal to 10 mol %, the amount of CTFE is at least 0.5 mol % and less than or equal to 1 mol %, the amount of HCFC-133b is at least 20 mol % and less than or equal to 24 mol %, the amount of HCFC-133 is at least 2 mol % and less than or equal to 5 mol %, based on the combined total moles of the HFC-123a, HFC-143, CTFE, HCFC-133b, and HCFC-133 in the product composition.

The combined amount of HFC-123a, HFC-143, CTFE, HCFC-133b, and HCFC-133 in the composition set forth in the preceding paragraph is at least 70 mol % and less than or equal to 100% of the total moles of organic components of the product composition in stream 206 from the reactor 204, while the combined amount of other components including undesired byproducts (e.g., HC-170, HFC-152a, HCFC-142 isomers (e.g., HCFC-142a, HCFC-142, and/or HCFC-142b), HCC-160, HFO-1123, HFC-143a, HF, HFC-152, and HFC-161) is greater than or equal to 0 mol % and less than 30 mol % of the total moles of organic components of the product composition in stream 206 from the reactor 204.

Process 2—Pathway I & Pathway II Step (i)—Product Stream Composition Post Processing

Referring to FIGS. 2a and 2b, bottom stream 210 from the separation unit 208 containing predominantly HCFC-123a is used as feed for either Step (ii) hydrogenation reaction of Process 2—Pathway I to produce HFC-143 in reactor 220, or Step (ii) dehydrochlorination reaction of Process 2—Pathway II to produce CTFE in vapor phase reactor 230a or liquid phase reactor 230b.

For example, for Step (i) of Process 2, both Pathways I and II, carried out using each catalyst/support combination (each row) in Table 3, referring to FIGS. 2a-2b, in the bottom stream 210 from the separation unit 208, the amount of R-123a is at least 80 wt % and less than or equal to 99.97 wt %, the amount of R-142 isomers is at least 0.01 wt % and less than or equal to 3 wt %, the amount of R-133 is at least 0.01 wt % and less than or equal to 15 wt %, and the amount of R-113 is at least 0.01 wt % and less than or equal to 2 wt %, based on the total amount of organic components of the final product composition in stream 210 from separation unit 208.

For example, for Step (i) of Process 2, both Pathways I and II, carried out using each catalyst/support combination (each row) in Table 3, referring to FIGS. 2a-2b, in the bottom stream 210 from the separation unit 208, the amount of R-123a is at least 90 wt % and less than or equal to 99.97 wt %, the amount of R-142 isomers is at least 0.01 wt % and less than or equal to 1 wt %, the amount of R-133 is at least 0.01 wt % and less than or equal to 8 wt %, and the amount of R-113 is at least 0.01 wt % and less than or equal to 1 wt %, based on the total amount of organic components of the final product composition in stream 210 from separation unit 208.

For example, for Step (i) of Process 2, both Pathways I and II, carried out using each catalyst/support combination (each row) in Table 3, referring to FIGS. 2a-2b, in the bottom stream 210 from the separation unit 208, the amount of R-123a is at least 99.5 wt % and less than or equal to 99.98 wt %, the amount of R-142 isomers is at least 0.01 wt % and less than or equal to 0.1 wt %, the amount of R-133 is at least 0.01 wt % and less than or equal to 0.3 wt %, and the amount of R-113 is at least 0 wt % and less than or equal to 0.1 wt %, based on the total amount of organic components of the final product composition in stream 210 from separation unit 208.

### Process 2—Pathway I Step (ii) Vapor Phase Reaction Catalyst

In the vapor phase dehydrochlorination reaction of Process 2 Pathway I Step (ii), the catalyst comprises, consists essentially of, or consists of (i) metal halides, (ii) halogenated metal oxides, or (iii) zero-valent metals/metal alloys.

In the vapor phase dehydrochlorination reaction of Process 2 Pathway I Step (ii), catalysts used may include metal halide catalysts including mono-, bi-, and tri-valent metal halides and their mixtures/combinations. Component metals include, but are not limited to, Cr³⁺, Fe³⁺, Mg²⁺, Ca²⁺, Ni²⁺, Zn²⁺, Pd²⁺, Li⁺, Na⁺, K⁺, and Cs⁺. Component halogens include, but are not limited to, F⁻, Cl⁻, Br⁻, and I⁻. Examples of preferred mono- or bi-valent metal halides include, but are not limited to, LiF, NaF, KF, CsF, MgF₂, CaF₂, LiCl, NaCl, KCl, and CsCl. The catalyst may be supported or unsupported, for example with one or a combination of catalyst supports discussed herein. The catalyst may be a combination of MgF₂ with one or more of CsCl, LiCl, NaCl, KCl, LiF, NaF, KF, and/or CsF. The CsCl, LiCl, NaCl, KCl, LiF, NaF, KF, and/or CsF may be present in an amount of about 5.0 wt. % to about 50 wt. %, based on the total weight of the catalyst.

In the vapor phase dehydrochlorination reaction of Process 2 Pathway I Step (ii), catalysts used may include halogenated metal oxide catalysts including halogenated mono-, bi-, and tri-valent metal oxides and their mixtures/combinations. Component metals include, but are not limited to, Cr³⁺, Fe³⁺, Mg²⁺, Ca²⁺, Ni²⁺, Zn²⁺, Pd²⁺, Li⁺, Na⁺, K⁺, and Cs⁺. Halogenation treatments can include any of those known in the prior art, particularly those that employ HF, F₂, HCl, Cl₂, HBr, Br₂, HI, and I₂ as the halogenation source. Examples of preferred halogenated mono- and bi-valent metal oxides include, but are not limited to, fluorinated or chlorinated MgO, fluorinated or chlorinated CaO, fluorinated or chlorinated Li₂O, fluorinated or chlorinated Na₂O, fluorinated or chlorinated K₂O, and fluorinated or chlorinated Cs₂O. The catalyst may be supported or unsupported, for example with one or a combination of catalyst supports discussed herein. The catalyst may include a combination of fluorinated MgO with one or more of a fluorinated Cs₂O, a fluorinated Li₂O, a fluorinated Na₂O, and/or a fluorinated K₂O. The fluorinated Cs₂O, fluorinated Li₂O, fluorinated Na₂O, and/or fluorinated K₂O may be present in an amount of about 5.0 to about 50 wt. %, based on the total weight of the catalyst.

In the vapor phase dehydrochlorination reaction of Process 2 Pathway I Step (ii), catalysts used may include zero-valent metal catalysts including neutral (e.g., zero valent) metals, metal alloys, and their mixtures. The zero valent metals may include, but are not limited to, Pd, Pt, Rh, Ru, Ir, Os, Fe, Co, Ni, Cu, Mo, Cr, Mn, and combinations of the foregoing as alloys or mixtures. The catalyst may be supported or unsupported. Useful supports include, but are not limited to MgO, fluorinated MgO, MgF₂, carbon materials for example activated carbons and carbon molecular sieves, α-Al₂O₃. The catalyst may include a combination of MgO with one or more of Pd, Pt, Ru, Rh, and/or Ir. The Pd, Pt, Ru, Rh, and/or Ir may be present in an amount of about 0.05 to about 5 wt. %, based on the total weight of the catalyst.

Non-limiting examples of metal alloys include stainless steel alloys (e.g., 304, 316, 316L, etc.), Monel alloys (e.g., 400, 401, 404, etc.), Inconel alloys (e.g., 600, 617, 625, 718, etc.), Hastelloy alloys (e.g., B-2, C-4, C-22, C-276, etc.), and Incoloy alloys (e.g., 800, 825, etc.). Preferred metal alloy catalysts include Inconel alloy (e.g., Inconel 625 alloy forming an inner surface of a metal alloy reactor vessel).

### Process 2—Pathway I Step (ii) Vapor Phase Reaction Temperature and Pressure

For a vapor phase dehydrochlorination reaction of Process 2 Pathway I Step (ii) using catalysts including metal halides, halogenated metal oxides, or zero-valent metals/metal alloys as described above, the reaction temperature of Step (ii) vapor phase dehydrochlorination reaction is as low as about 400 °C, about 425 °C, about 450 °C, about 475 °C, or as high as about 525 °C, about 550 °C, about 575 °C, about 600 °C, or within any range encompassed by two of the foregoing values as endpoints, for example from about 400 °C to about 600 °C, or from about 425 °C to about 575 °C, for example. The temperature is preferably from about 475 °C to about 525 °C.

A variety of reaction pressures may be used, for example superatmospheric, atmospheric, and subatmospheric. In certain aspects, atmospheric pressure is preferred.

### Process 2—Pathway I Step (ii) Vapor Phase Reaction—Selectivity to CTFE

As demonstrated by the Examples herein, for a vapor phase dehydrochlorination reaction of Process 2 Pathway I Step (ii) using catalysts including metal halides, halogenated metal oxides, or zero-valent metals/metal alloys as described above, the Step (ii) vapor phase dehydrochlorination reaction may achieve a selectivity to the desired product CTFE of at least about 70%, at least about 80%, at least about 90%, and for each of the foregoing, less than or equal to 100%, or within any range encompassed by two of the foregoing values as endpoints, for example from about 70% to about 100%, or from about 80% to about 100%, based on total moles of the organic components of the composition.

### Process 2—Pathway I Step (ii) Vapor Phase Reaction—Conversion of HCFC-123a

As demonstrated by the Examples herein, for a vapor phase dehydrochlorination reaction of Process 2 Pathway I Step (ii) using catalysts including metal halides, halogenated metal oxides, or zero-valent metals/metal alloys as described above, the Step (ii) vapor phase dehydrochlorination reaction may achieve a conversion of at least about 5%, at least about 10%, or at least about 15%, and for each of the foregoing, less than or equal to 100%, or within any range encompassed by two of the foregoing values as endpoints, for example from about 5% to about 100%, or from about 10% to about 100%, based on total moles of the organic components of the composition.

### Process 2—Pathway I Step (ii) Vapor Phase Reaction—Post Processing

Inside reactor 230a, referring to FIG. 2a, where the reaction system is Process 2—Pathway II, the HCFC-123a undergoes the Step (ii) Vapor Phase dehydrochlorination reaction to produce a second product composition comprising CTFE. Stream 232a including a second product composition enters separation unit 234 (e.g., columns, scrubbers, adsorbers, absorbers, membrane separation units, flash vessels, vaporizers, etc.) for producing stream 236a including predominantly CTFE and recycle stream 238a.

For example, for Pathway I Step (ii) vapor phase reaction of Process 2 carried out using suitable catalysts described herein, referring to FIG. 2a, in the final product stream 236a from separation unit 234, the amount of CTFE is at least 90 wt % and less than or equal to 99.95 wt %, the amount of R-1122 is at least 0.01 wt % and less than or equal to 2 wt %, the amount of R-133 is at least 0.01 wt % and less than or equal to 2 wt %, the amount of dimers is at least 0.01 wt % and less than or equal to 2 wt %, the amount of R-123a is at least 0.01 wt % and less than or equal to 2 wt %, and the amount of R-1123 is at least 0.01 wt % and less than or equal to 2 wt %, based on the total amount of organic components of the final product composition in stream 236a from separation unit 234.

For example, for Pathway I Step (ii) vapor phase reaction of Process 2 carried out using suitable catalysts described herein, referring to FIG. 2a, in the final product stream 236a from separation unit 234, the amount of CTFE is at least 95 wt % and less than or equal to 99.95 wt %, the amount of R-1122 is at least 0.01 wt % and less than or equal to 1 wt %, the amount of R-133 is at least 0.01 wt % and less than or equal to 1 wt %, the amount of dimers is at least 0.01 wt % and less than or equal to 1 wt %, the amount of R-123a is at least 0.01 wt % and less than or equal to 1 wt %, and the amount of R-1123 is at least 0.01 wt % and less than or equal to 1 wt %, based on the total amount of organic components of the final product composition in stream 236a from separation unit 234.

For example, for Pathway I Step (ii) vapor phase reaction of Process 2 carried out using suitable catalysts described herein, referring to FIG. 2a, in the final product stream 236a from separation unit 234, the amount of CTFE is at least 99.7 wt % and less than or equal to 99.95 wt %, the amount of R-1122 is at least 0.01 wt % and less than or equal to 0.05 wt %, the amount of R-133 is at least 0.01 wt % and less than or equal to 0.05 wt %, the amount of dimers is at least 0.01 wt % and less than or equal to 0.1 wt %, the amount of R-123a is at least 0.01 wt % and less than or equal to 0.05 wt %, and the amount of R-1123 is at least 0.01 wt % and less than or equal to 0.05 wt %, based on the total amount of organic components of the final product composition in stream 236a from separation unit 234.

### Process 2—Pathway I Step (ii) Liquid Phase Reaction Base

In the liquid phase dehydrochlorination reaction of Process 2 Pathway I Step (ii), HCFC-123a can be reacted with a suitable base to directly form CFO-1113 and other byproducts. Suitable bases include, but are not limited to, potassium hydroxide (KOH), sodium hydroxide (NaOH), calcium oxide (CaO), and/or calcium hydroxide (Ca(OH)₂).

### Process 2—Pathway I Step (ii) Liquid Phase Reaction Molar Ratio

In the liquid phase dehydrochlorination reaction of Process 2 Pathway I Step (ii), the HCFC-123a containing feed stream (e.g., stream 210 in FIG. 2b) and the base containing feed stream (e.g., stream 218b in FIG. 2b) can be provided to the reactor (e.g., reactor 230b in FIG. 2b) in rates and amounts that result in a mole ratio of base to HCFC-123a in the reactor of less that about 0.5:1 up to about to 3:1, preferably from about 1:1 to about 1.5:1.

The base containing feed stream can be an aqueous mixture, and can contain the base at any suitable strength for producing the desired reaction. For example, when KOH is used as the base, the base containing feed stream can contain base at a strength from about 5 wt. % to about 50 wt. %, preferably from about 20 wt. % to about 40 wt. %. Optionally, a phase transfer agent or catalyst, for example, alcohol and Aliquat®336 (e.g., C₂₅H₅₄ClN) can be added to the reaction in the at least one reactor, which can enhance the reaction rate.

### Process 2—Pathway I Step (ii) Liquid Phase Reaction Temperature

For a liquid phase dehydrochlorination reaction of Process 2 Pathway I Step (ii) using bases including KOH, NaOH, CaO, and/or Ca(OH)₂ as described above, the reaction temperature of Step (ii) liquid phase dehydrochlorination reaction is as low as about 40 °C, about 45 °C, about 50 °C, or as high as about 90 °C, about 95 °C, about 100 °C, or within any range encompassed by two of the foregoing values as endpoints, for example from about 40 °C to about 100 °C, or from about 45 °C to about 90 °C, for example.

### Process 2—Pathway I Step (ii) Liquid Phase Reaction Product

For a liquid phase dehydrochlorination reaction of Process 2 Pathway I Step (ii) using bases including KOH, NaOH, CaO, and/or Ca(OH)₂ as described above, the reaction product vapors formed during the reaction that occurs in the reactor (e.g., reactor 230b in FIG. 2b) can include CTFE, HCFC-123a, and water vapor. The reaction product vapors can also contain at least some entrained KOH and KCl when KOH is used as the reactant base.

As demonstrated by the Examples herein, for a liquid phase dehydrochlorination reaction of Process 2 Pathway I Step (ii) using bases including KOH, NaOH, CaO, and/or Ca(OH)₂ as described above, the Step (ii) liquid phase dehydrochlorination reaction may achieve a selectivity to the desired product CTFE of at least about 70%, at least about 80%, at least about 90%, and for each of the foregoing, less than or equal to 100%, or within any range encompassed by two of the foregoing values as endpoints, for example from about 70% to about 100%, or from about 80% to about 100%, based on total moles of the organic components of the composition.

### Process 2—Pathway I Step (ii) Liquid Phase Reaction—Post Processing

Inside reactor 230b, referring to FIG. 2b, where the reaction system is Process 2—Pathway II, the HCFC-123a undergoes the Step (ii) Liquid Phase dehydrochlorination reaction to produce a second product composition comprising CTFE. Stream 232b including a second product composition enters separation unit 234 (e.g., columns, scrubbers, adsorbers, absorbers, membrane separation units, flash vessels, vaporizers, etc.) for producing stream 236b including predominantly CTFE and recycle stream 238b.

For example, for Pathway I Step (ii) liquid phase reaction of Process 2 carried out using suitable bases described herein, referring to FIG. 2b, in the final product stream 236b from separation unit 234, the amount of CTFE is at least 90 wt % and less than or equal to 99.95 wt %, the amount of R-1122 is at least 0.01 wt % and less than or equal to 2 wt %, the amount of R-133 is at least 0.01 wt % and less than or equal to 2 wt %, the amount of dimers is at least 0.01 wt % and less than or equal to 2 wt %, the amount of R-123a is at least 0.01 wt % and less than or equal to 2 wt %, and the amount of R-1123 is at least 0.01 wt % and less than or equal to 2 wt %, based on the total amount of organic components of the final product composition in stream 236b from separation unit 234.

For example, for Pathway I Step (ii) liquid phase reaction of Process 2 carried out using suitable bases described herein, referring to FIG. 2b, in the final product stream 236b from separation unit 234, the amount of CTFE is at least 95 wt % and less than or equal to 99.95 wt %, the amount of R-1122 is at least 0.01 wt % and less than or equal to 1 wt %, the amount of R-133 is at least 0.01 wt % and less than or equal to 1 wt %, the amount of dimers is at least 0.01 wt % and less than or equal to 1 wt %, the amount of R-123a is at least 0.01 wt % and less than or equal to 1 wt %, and the amount of R-1123 is at least 0.01 wt % and less than or equal to 1 wt %, based on the total amount of organic components of the final product composition in stream 236b from separation unit 234.

For example, for Pathway I Step (ii) liquid phase reaction of Process 2 carried out using suitable bases described herein, referring to FIG. 2b, in the final product stream 236b from separation unit 234, the amount of CTFE is at least 99.7 wt % and less than or equal to 99.95 wt %, the amount of R-1122 is at least 0.01 wt % and less than or equal to 0.05 wt %, the amount of R-133 is at least 0.01 wt % and less than or equal to 0.05 wt %, the amount of dimers is at least 0.01 wt % and less than or equal to 0.1 wt %, the amount of R-123a is at least 0.01 wt % and less than or equal to 0.05 wt %, and the amount of R-1123 is at least 0.01 wt % and less than or equal to 0.05 wt %, based on the total amount of organic components of the final product composition in stream 236b from separation unit 234.

### Process 2—Pathway II Step (ii) Catalysts

In the hydrogenation reaction of Process 2 Pathway II Step (ii), the catalyst used may be selected from the same list as described above regarding Step (i) hydrogenation reaction of Process 2. For example, the catalyst may comprise a metal including but not limited to palladium, platinum, rhodium, ruthenium, iron, cobalt or nickel. The catalyst active to catalyze the reaction may preferably be palladium metal (Pd), platinum metal (Pt), or a combination of palladium metal and platinum metal.

### Process 2—Pathway II Step (ii) Catalyst Support

In the hydrogenation reaction of Process 2 Pathway II Step (ii), the catalyst may be supported on a suitable support, including but not limited to carbon or alumina (aluminum oxide—Al₂O₃). The carbon may be activated carbon. The alumina may be alpha(α) alumina, theta(θ) alumina, delta(δ) alumina, or gamma(γ) alumina. The supported catalyst may be produced by impregnation of any of the suitable supports with a solution of a compound of the desired metal constituent. The support may also be in the form of pellets. After the impregnation step, the solvent may be removed using heat or under vacuum resulting in a solid mass which can be further dried, calcined, and reduced to form active metal catalyst.

In the hydrogenation reaction of Process 2 Pathway II Step (ii), the catalyst may be palladium on a carbon support, may be platinum on a carbon support, may be rhodium on a carbon support, and/or may be palladium, platinum, or rhodium on an alumina support. The catalyst may be palladium on a carbon support or palladium on an alpha alumina support.

In the hydrogenation reaction of Process 2 Pathway II Step (ii), the metal catalysts supported on various catalyst supports used may be the same as Step (i) hydrogenation reaction of Process 2 as listed in Table 3 above.

### Process 2—Pathway II Step (ii) Catalyst Loading

For each catalyst/support combination (each row) in Table 3 used in the hydrogenation reaction of Process 2 Pathway II Step (ii), the amount of metal loading on the support is from as little as about 0.01 wt. %, about 0.05 wt. %, about 0.1 wt. %, about 0.2 wt. %, about 0.3 wt. %, about 0.4 wt. %, about 0.5 wt. %, about 1 wt. %, or as great as about 2 wt. %, about 3 wt. %, about 5 wt. %, about 10 wt. %, about 20 wt. %, about 30 wt. %, about 40 wt. %, about 50 wt. %, based on a total weight of the catalyst and support, or within any range encompassed by two of the foregoing values as endpoints, for example, from about 0.01 wt. % to about 20 wt. %, from about 0.01 wt. % to about 10 wt. %, from about 0.1 wt. % to about 10 wt. %, 0.2 wt. % to about 10 wt. %, from about 0.5 wt. % to about 5 wt. %, from about 1 wt. % to about 5 wt. %, from about 1 wt. % to about 4 wt. %, or about 2 wt. % to about 4 wt. %, based on a total weight of the catalyst and support. For supported noble metal catalysts including but not limited to Pd or Pt, the metal loading is from about 0.01 wt. % to about 5 wt. % based on a total weight of the catalyst and support.

### Process 2—Pathway II Step (ii) Catalyst BET Surface Area

For each catalyst/support combination (each row) in Table 3 used in the hydrogenation reaction of Process 2 Pathway II Step (ii), the BET surface area may be as low as about 0.5 m²/g, about 1 m²/g, about 3 m²/g, about 5 m²/g, about 10 m²/g, about 15 m²/g, about 20 m²/g², about 30 m²/g, about 40 m²/g, about 50 m²/g, about 100 m²/g, about 200 m²/g, or as high as about 250 m²/g, about 300 m²/g, about 400 m²/g, about 500 m²/g, about 600 m²/g, about 700 m²/g m², about 800 m²/g, about 900 m²/g, about 1000 m²/g, about 2000 m²/g, about 3000 m²/g, or within any range encompassed by any of the foregoing values as endpoints, from example, from about 0.5 m²/g to about 3000 m²/g, from about 1 m²/g to about 2000 m²/g, from about 1000 m²/g to about 2000 m²/g, from about 0.5 m²/g to about 500 m²/g, from about 0.5 m²/g to about 300 m²/g, from about 0.5 m²/g to about 5 m²/g, or from about 200 m²/g to about 300 m²/g.

### Process 2—Pathway II Step (ii) Catalyst Pretreatment

For each catalyst/support combination (each row) in Table 3 used in the hydrogenation reaction of Process 2 Pathway II Step (ii), the catalyst may be pretreated by a variety of methods to improve its performance and effectiveness in the reaction. For example, the catalyst may be dried at elevated temperatures, as low as about 200 °C, about 250 °C, about 300 °C, about 350 °C, about 360 °C, about 370 °C, or as high as about 380 °C, about 390 °C, about 400 °C, about 450 °C, about 500 °C, about 600 °C, about 700 °C, or within any range encompassed by two of the foregoing values as endpoints, for example from about 200 °C to about 400 °C, from about 200 °C to about 350 °C, from about 250 °C to about 350 °C, from about 250 °C to about 300 °C, or from about 260 °C to about 300 °C.

### Process 2—Pathway II Step (ii) Temperature

For reactions using each catalyst/support combination (each row) in Table 3, the reaction temperature of the hydrogenation reaction of Process 2 Pathway II Step (ii) is as low as about 100 °C, about 125 °C, about 150 °C, about 200 °C, about 205 °C, about 210 °C, about 215 °C, about 220 °C, about 225 °C, about 230 °C, about 235 °C, about 240 °C, about 245 °C, about 250 °C, about 255 °C, or as high as about 260 °C, about 265 °C, about 270 °C, about 275 °C, about 280 °C, about 285 °C, about 290 °C, about 295 °C, about 300 °C, about 305 °C, about 310 °C, about 315 °C, about 320 °C, about 325 °C, about 350 °C, about 400 °C, or within any range encompassed by any two of the foregoing values as endpoints. For example, the reaction temperature is from about 100 °C to about 400 °C, or from about 125 °C to about 350 °C. The temperature is preferably from about 100 °C to about 350 °C, and more preferably from about 200 °C to about 300 °C.

### Process 2—Pathway II Step (ii) Contact Time

For reactions using each catalyst/support combination (each row) in Table 3, the contact time of the reactants with the catalyst (Pd, Pt) in the hydrogenation reaction of Process 2 Pathway II Step (ii) is as little as about 0.1 second, about 1 second, about 2 seconds, about 5 seconds, about 10 seconds, about 15 seconds, about 20 seconds, or as long as about 25 seconds, about 30 seconds, about 40 seconds, about 50 seconds, about 60 seconds, about 80 seconds, about 120 seconds, or within any range encompassed by two of the foregoing values as endpoints, for example from about 0.1 seconds to about 120 seconds, from about 1 second to about 60 seconds, from about 5 seconds to about 50 seconds, from about 10 seconds to about 40 seconds, from about 15 seconds to about 30 seconds, or about 20 seconds to about 25 seconds. For example, the contact time is from about 1 second to about 60 seconds.

### Process 2—Pathway II Step (ii) Pressure

For reactions using each catalyst/support combination (each row) in Table 3, the pressure inside which reactor the hydrogenation reaction of Process 2 Pathway II Step (ii) takes place is as little as about 1 psig, about 3 psig, about 5 psig, about 10 psig, about 15 psig, about 20 psig, about 30 psig, about 35 psig or about 40 psig, or as great as about 90 psig, about 100 psig, about 120 psig, about 150 psig, about 200 psig or about 250 psig, about 300 psig, or within any range encompassed by two of the foregoing values as endpoints, for example from about 1 psig to about 300 psig, from about 3 psig to about 250 psig, from about 5 psig to about 200 psig, from about 10 psig to about 150 psig, from about 15 psig to about 120 psig, from about 20 psig to about 100 psig, from about 30 psig to about 90 psig, or from about 35 psig to about 40 psig. For example, the pressure is from about 10 psig to about 200 psig.

### Process 2—Pathway II Step (ii) Hydrogen Mole Ratio

For reactions using each catalyst/support combination (each row) in Table 3 used in the hydrogenation reaction of Process 2 Pathway II Step (ii), the mole ratio of hydrogen to HCFC-123a is as little about 2:1, about 3:1, about 4:1, about 5:1, about 5.5:1 or as great as about 6:1, about 6.5:1, about 7.5:1 or about 8:1, about 12:1, about 15:1, or about 20:1, for example, or within any range encompassed by two of the foregoing values as endpoints. The mole ratio of hydrogen to HCFC-123a is preferably from about 3:1 about to 15:1, and more preferably from about 4:1 to about 10:1.

### Process 2—Pathway II Step (ii) Products—Selectivity to HFC-143

As demonstrated by the Examples herein, for reactions using each catalyst/support combination (each row) in Table 3, the hydrogenation reaction of Process 2 Pathway II Step (ii) may achieve a selectivity to the desired product 1,1,2-trifluoroethane (HFC-143) of at least about 20%, at least about 30%, at least about 40%, at least about 50%, at least about 60%, at least about 70%, at least about 80%, at least about 90%, and for each of the foregoing, less than or equal to 100%, or within any range encompassed by two of the foregoing values as endpoints, for example from about 20% to about 90%, from about 30% to about 80%, from about 40% to about 70%, or about 50% to about 60%, based on total moles of the organic components of the composition.

Process 2—Pathway II Step (ii)—Conversion of HCFC-123a

For the hydrogenation reaction of Process 2 Pathway II Step (ii) carried out using each catalyst/support combination (each row) in Table 3, the reaction may achieve a conversion of HCFC-123a of at least about 40%, at least about 50%, at least about 60%, at least about 70%, or at least about 80%, and for each of the foregoing, less than or equal to 100%, or within any range encompassed by two of the foregoing values as endpoints, for example from about 50% to about 100%, or from about 60% to about 100%, based on total moles of the organic components of the composition.

Process 2—Pathway II Step (ii)—Product Stream from Reactor

Referring again to FIGS. 2a-2b, the hydrogenation reaction of Process 2 Pathway II Step (ii) inside reactor 220 produces a product stream 222 (before any post processing steps) comprising the target product 1,1,2-trifluoroethane (HFC-143), recyclable intermediates including but not limited to 1-chloro-1,1,2-trifluoroethane (HCFC-133b), and/or 1-chloro-1,2,2-trifluoroethane (HCFC-133), unreacted starting materials including but not limited to hydrogen (H₂) and 1,2-dichloro-1,1,2-trifluoroethane (HCFC-123a), and other byproducts including but not limited to ethane (HC-170), 1,1-difluoroethane (HFC-152a), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), chloroethane (HCC-160), 1,1,1-trifluoroethane (HFC-143a), hydrogen chloride (HCl), and hydrogen fluoride (HF).

For the hydrogenation reaction of Process 2 Pathway II Step (ii) carried out using each catalyst/support combination (each row) in Table 3, referring to FIGS. 2a-2b, the amount of unreacted HCFC-123a in the product composition in stream 222 from the reactor 220, is greater than or equal to 0.1 mol % and yet less than 10 mol %, less than 20 mol %, or less than 30 mol %, for example, based on total moles of organic components of the composition.

For the hydrogenation reaction of Process 2 Pathway II Step (ii) carried out using each catalyst/support combination (each row) in Table 3, referring to FIGS. 2a-2b, the amount of 1,1,2-trifluoroethane (HFC-143) in the product composition in stream 222 from the reactor 220 is at least 40 mol %, at least 50 mol %, or at least 60 mol %, and for each of the foregoing, less than or equal to 100 mol %, for example, based on total moles of organic components of the composition.

For the hydrogenation reaction of Process 2 Pathway II Step (ii) carried out using each catalyst/support combination (each row) in Table 3, referring to FIGS. 2a-2b, the amount of 1-chloro-1,1,2-trifluoroethane (HCFC-133b) in the product composition in stream 122 from the reactor 120, if present, is greater than or equal to 0.5% and yet less than 50 mol %, less than 45 mol %, less than 40 mol %, or less than 35 mol %, for example, based on total moles of organic components of the composition.

For the hydrogenation reaction of Process 2 Pathway II Step (ii) carried out using each catalyst/support combination (each row) in Table 3, referring to FIGS. 2a-2b, the amount of 1-chloro-1,2,2-trifluoroethane (HCFC-133) in the product composition in stream 122 from the reactor 120, if present, is greater than or equal to 0.1 mol % and yet less than 10 mol %, less than 5 mol %, or less than 3 mol %, for example, based on total moles of organic components of the composition.

For the hydrogenation reaction of Process 2 Pathway II Step (ii) carried out using each catalyst/support combination (each row) in Table 3, referring to FIGS. 2a-2b, the total amount of 1,1,2-trifluoroethane (HFC-143), 1-chloro-1,1,2-trifluoroethane (HCFC-133b), and 1-chloro-1,2,2-trifluoroethane (HCFC-133) in the product composition in stream 222 from the reactor 220 is at least 80 mol %, at least 85 mol %, or at least 90 mol %, and for each of the foregoing, less than or equal to 100 mol %, for example, based on total moles of organic components of the composition.

For example, for the hydrogenation reaction of Process 2 Pathway II Step (ii) carried out using each catalyst/support combination (each row) in Table 3, referring to FIGS. 2a-2b, in the product composition in stream 222 from the reactor 220, the amount of 1,1,2-trifluoroethane (HFC-143) is at least 50 mol % and less than or equal to 99.4 mol %, the amount of 1-chloro-1,1,2-trifluoroethane (HCFC-133b) is at least 0.5 mol % and less than or equal to 45 mol %, the amount of 1-chloro-1,2,2-trifluoroethane (HCFC-133) is at least 0.1 mol % and less than or equal to 5 mol %, based on the combined total moles of the HFC-143, HCFC-133b, and HCFC-133 in the product composition.

The combined amount of HFC-143, HCFC-133b, and HCFC-133 in the composition set forth in the preceding paragraph is at least 60 mol % and less than or equal to 100% of the total moles of organic components of the product composition in stream 222 from the reactor 220, while the combined amount of other components including unreacted HCFC-123a and undesired byproducts (e.g., HFC-143a, HC-170, HCC-160, HFC-152a, and HCFC-142 isomers) is greater than or equal to 0 mol % and less than 40 mol % of the total moles of organic components of the product composition in stream 108 from the reactor 106.

For example, for the hydrogenation reaction of Process 2 Pathway II Step (ii) carried out using each catalyst/support combination (each row) in Table 3, referring to FIGS. 2a-2b, in the product composition in stream 222 from the reactor 220, the amount of 1,1,2-trifluoroethane (HFC-143) is at least 55 mol % and less than or equal to 99.4 mol %, the amount of 1-chloro-1,1,2-trifluoroethane (HCFC-133b) is at least 0.5 mol % and less than or equal to 40 mol %, and the amount of 1-chloro-1,2,2-trifluoroethane (HCFC-133) is at least 0.1 mol % and less than or equal to 5 mol %, based on the combined total moles of the HFC-143, HCFC-133b, and HCFC-133 in the product composition.

The combined amount of HFC-143, HCFC-133b, and HCFC-133 in the composition set forth in the preceding paragraph is at least 70 mol % and less than or equal to 100% of the total moles of organic components of the product composition in stream 222 from the reactor 220, while the combined amount of other components including unreacted HCFC-123a and undesired byproducts (e.g., HFC-143a, HC-170, HCC-160, HFC-152a, and HCFC-142 isomers) is greater than or equal to 0 mol % and less than 30 mol % of the total moles of organic components of the product composition in stream 108 from the reactor 106.

### V. Process 3

Process 3—Step (i)—Reaction Feed

Product compositions as described above comprising HFC-143 may be further processed or purified to obtain a reactant stream of HFC-143 to be provided to Step (i). The reactant composition provided to Step (i) comprises liquid 1,1,2-trifluoroethane (HFC-143) having a purity of at least 50 wt. %, 60 wt. %, 95.0 wt. %, 99.0 wt. %, 99.5 wt. %, 99.9 wt. %, or within any range defined by any two of the foregoing values as endpoints as based on total weight of organic components. In other embodiments, the reactant composition provided to Step (i) comprises liquid 1,1,2-trifluoroethane (HFC-143) and one or more additional organic components, including one or more intermediates, undesired byproducts, or reaction byproducts, such as HCFC-133b, HCFC-133, HCFC-123a, HFC-152a, HFO-1123, HFO-1132a, HFO-1132(Z), HFO-1132(E), or combinations thereof. Here, the reactant composition may be introduced batchwise or semi-batchwise into a reaction vessel optionally equipped with any one of or combination of agitation, a heating and cooling jacket, and an integrated reactor distillation column and condenser, thereby permitting reaction and simultaneous separation of one or more volatile components during Step (ii).

Process 3—Step (i)—Reaction Base

In the liquid phase dehydrofluorination reaction of Step (ii), HFC-143 can be reacted with a suitable base to form HFO-1132 (Z), HFO-1132(Z), HFO-1132a, and other byproducts. Suitable bases include, but are not limited to, potassium hydroxide (KOH), sodium hydroxide (NaOH), calcium oxide (CaO), and/or calcium hydroxide (Ca(OH)₂).

Process 3—Step (i)—Reaction Temperature

The reactants may reach a target temperature in the preheater 115 or in the reactor 114. In one example, the preheater 115 brings reactants up to a first temperature and the reactor 114 maintains the reaction at the first temperature. In another example, the reactor 114 brings the reactants up to a second temperature and maintains the reaction at the second temperature, wherein the second temperature is greater than or less than the first temperature. The reaction temperature of the liquid phase dehydrofluorination reaction of Step (ii) may be as low as about 90 °C, about 95 °C, about 100 °C, about 105 °C, about 110 °C, about 115 °C, or as high as about 120 °C, about 125 °C, about 130 °C, about 135 °C, about 140 °C, about 150 °C, or within any range encompassed by two of the foregoing values as endpoints, for example from about 90 °C to about 150 °C, or from about 95 °C to about 140 °C. The temperature may be preferably from about 95 °C to about 130 °C, and more preferably from about 100 °C to about 120 °C.

Process 3—Step (i)—Base to Reactant Mole Ratio

The mole ratio of base to reactant (HFC-143, HCFC-133b, HCFC-133, and HCFC-123a) of the liquid phase dehydrofluorination reaction of Step (ii) may be as low as about 0.1, about 0.2, about 0.3, about 0.4, about 0.5, about 0.6, about 0.7, about 0.8, about 0.9, about 1.0, or as high as about 1.1, about 1.2, about 1.3, about 1.4, about 1.5, about 1.6, about 1.7, about 1.8, about 1.9, about 2.0, about 3.0, about 4.0, about 5.0, or within any range encompassed by two of the foregoing values as endpoints, for example from about 0.1 to about 2.0, or from about 0.2 to about 1.6, preferably from about 1 to about 2. The mole ratio of base to reactant (HFC-143, HCFC-133b, HCFC-133, and HCFC-123a) may preferably be from about 0.8 to about 1.8, more preferably from about 1.0 to about 1.5, and most preferably from about 1.0 to about 1.2. Specific examples of additional suitable ranges are set forth below in Table 8. The numerical ranges set forth in Table 8 below are understood to be prefaced by “about”.

The base containing feed stream can be an aqueous mixture and can contain the base at any suitable strength for producing the desired reaction. For example, when KOH is used as the base, the base containing feed stream can contain base at a strength from about 5 wt. % to about 50 wt. %, preferably from about 20 wt. % to about 45 wt. %. Optionally, a phase transfer agent or catalyst, for example, alcohol and Aliquat®336 (C₂₅H₅₄ClN) can be added to the reaction in the at least one reactor, which can enhance the reaction rate. Other phase transfer catalysts that can be used include, but not limited to, quaternary ammonium salts, quaternary phosphonium salts, quaternary arsonium salts, sulfonium salts, crown ethers and similar molecules. A quaternary ammonium salt or a quaternary phosphonium salt is more preferred. If a phase transfer catalyst is used, the base containing feed stream may comprise phase transfer catalyst in an amount greater than about 0.01 wt. %, about 0.02 wt. %, about 0.03 wt. %, about 0.04 wt. %, about 0.05 wt. %, or less than about 0.06 wt. %, about 0.07 wt. %, about 0.08 wt. %, about 0.09 wt. %, about 0.1 wt. %, about 1 wt. %, or within any range encompassed by two of the foregoing values as endpoints, for example from about 0.01 wt. % to about 1 wt. %, preferably from about 0.01 wt. % to about 0.1 wt. %, more preferably from about 0.03 wt. % to about 0.08 wt. %.

Process 3—Step (i)—Base Weight Percentage

The weight percentage of the base used for the liquid phase dehydrochlorination reaction of Step (i) may be as low as about 5 wt. %, about 10 wt. %, about 15 wt. %, about 20 wt. %, about 25 wt. %, or as high as about 30 wt. %, about 35 wt. %, about 40 wt. %, about 45 wt. %, about 50 wt. %, about 55 wt. %, about 60 wt. %, or within any range encompassed by two of the foregoing values as endpoints, for example from about 5 wt. % to about 50 wt. %, based on total weight of the base. The weight percentage of the base may preferably be from about 10 wt. % to about 60 wt. %, and more preferably from about 10 wt. % to about 45 wt. %, based on total weight of the base.

Process 3—Step (i)—Reaction Conditions

A summary of the preferred base, temperatures, base weight percentages, and base (OH⁻) to reactant (e.g., HFC-143, HCFC-133b, HCFC-133, and HCFC-123a) mole ratio as discussed above are summarized in Table 10 below. The numerical ranges set forth in Table 4 below are understood to be prefaced by “about”.

TABLE 4
Summary of Preferred Step (ii) Reaction Conditions

| | Base Weight Percentage | | |
|---|---|---|---|
| Base | (wt. %) | Mole ratio* | Temperature (°C) |
| KOH | 5-60 | 0.1-4 | 90-150 |
| KOH | 5-60 | 0.1-4 | 95-140 |
| KOH | 5-60 | 0.1-4 | 100-130 |
| KOH | 5-60 | 0.1-4 | 100-120 |
| KOH | 5-60 | 0.5-3 | 90-150 |
| KOH | 5-60 | 0.5-3 | 95-140 |
| KOH | 5-60 | 0.5-3 | 100-130 |
| KOH | 5-60 | 0.5-3 | 100-120 |
| KOH | 5-60 | 1-2 | 90-150 |
| KOH | 5-60 | 1-2 | 95-140 |
| KOH | 5-60 | 1-2 | 100-130 |
| KOH | 5-60 | 1-2 | 100-120 |
| KOH | 10-55 | 0.1-4 | 90-150 |
| KOH | 10-55 | 0.1-4 | 95-140 |
| KOH | 10-55 | 0.1-4 | 100-130 |
| KOH | 10-55 | 0.1-4 | 100-120 |
| KOH | 10-55 | 0.5-3 | 90-150 |
| KOH | 10-55 | 0.5-3 | 95-140 |
| KOH | 10-55 | 0.5-3 | 100-130 |
| KOH | 10-55 | 0.5-3 | 100-120 |
| KOH | 10-55 | 1-2 | 90-150 |
| KOH | 10-55 | 1-2 | 95-140 |
| KOH | 10-55 | 1-2 | 100-130 |
| KOH | 10-55 | 1-2 | 100-120 |

*Mole ratio denotes the mole ratio of base (OH⁻) to reactant (e.g., HFC-143, HCFC-133b, HCFC-133, and HCFC-123a)

Process 3—Step (i)—Conversion of HFC-143 As demonstrated by the Examples herein, for the liquid phase dehydrofluorination reaction of Step (ii), the reaction may achieve a conversion of HFC-143 of at least 25%, at least about 30%, at least about 35%, or at least about 40%, and for each of the foregoing, less than or equal to 100%, or within any range encompassed by two of the foregoing values as endpoints, for example from about 25% to about 100%, or from about 35% to about 100%, based on total moles of the organic components of the composition. The reaction may be considered complete by an increase in condenser temperature to approximately 60-80 °C, reflecting depletion of volatile components in the reactor overhead.

Process 3—Step (i)—Selectivity

As demonstrated by the Examples herein, for the liquid phase dehydrofluorination reaction of Step (ii), the reaction may achieve a selectivity towards HFO-1132 isomers of at least 35%, at least about 45%, at least about 55%, or at least about 65%, and for each of the foregoing, less than or equal to 100%, or within any range encompassed by two of the foregoing values as endpoints, for example from about 35% to about 100%, or from about 65% to about 100%, based on total moles of the organic components of the composition.

Process 3—Step (i)—Side products

At least one side reaction may occur during the dehydrofluorination reaction of Step (ii), which may create various side products alongside the formation of HFO-1132 isomers. The side products may include at least one of HFO-1132a, R-1123, R-1113, R-1112, CFCl═CHF (R-1122a), CF₂═CHCl (R-1122), CHCl═CHF (R-1131), CH2≡CHF (R-1141), CF≡CH, CCl≡CH, difluoroethanol, 2,2-difluoroethanol, dimers/trimers/oligomers, and KF.

Process 3—Step (ii)—Process

The 1,2-difluoroethylene (HFO-1132) obtained in Step (i) above may be produced as a mixture containing both the trans-1,2-difluoroethylene (HFO-1132E) and cis-1,2-difluoroethylene (HFO-1132Z) isomers. Step (ii) may be an isomerization of HFO-1132Z into HFO-1132E.

The isomerization of HFO-1132Z may take place in the presence of an optional catalyst, such as reactive monomers like chlorotrifluoroethylene (CTFE); alumina (Al₂O₃); chromia (Cr₂O₃); zeolites; fluorinated metal oxides; basic catalysts; and halide-promoted catalysts. An amount of catalyst may be added to the isomerization reaction in an amount from as little as 1 wt. %, 2 wt. %, 4 wt. %, 6 wt. %, 8 wt. %, or 10 wt. % to as great as 12 wt. %, 13 wt. %, 16 wt. %, 18 wt. %, or 20 wt. %, or any range using any two of the foregoing values as endpoints, such as 1 wt. % to 20 wt. %, 2 wt. % to 18 wt. %, 4 wt. % to 16 wt. %, 6 wt. % to 13 wt. %, 8 wt. % to 12 wt. %, or 10 wt. % to 12 wt. %, based on total weight of organic components of the reactant composition.

Further, an optional diluent may be added to the isomerization reaction. Suitable diluents may comprise nitrogen gas or argon gas.

In step (ii), the cis-1,2-difluoroethylene (HFO-1132Z) isomer may be converted to the trans-1,2-difluoroethylene (HFO-1132E) isomer either by exposure to heat and/or a catalyst to yield a crude product composition comprising at least 5 wt. % trans-1,2-difluoroethylene (HFO-1232E), at least 10 wt. % trans-1,2-difluoroethylene (HFO-1232E), at least 18 wt. % trans-1,2-difluoroethylene (HFO-1232E), at least 20 wt. % trans-1,2-difluoroethylene (HFO-1232E), at least 30 wt. % trans-1,2-difluoroethylene (HFO-1232E), or greater.

Crude product composition 126 may comprise an amount of cis-1,2-difluoroethylene (HFO-1132Z) from as little as about 10 wt. %, about 15 wt. %, about 20 wt. %, about 25 wt. %, or about 30 wt. % to as great as about 35 wt. %, about 40 wt. %, about 50 wt. %, about 60 wt. %, about 66 wt. %, about 70 wt. %, or any range using any two of the forgoing values as endpoints, such as from about 10 wt. % to 70 wt. %, from about 15 wt. % to 66 wt. %, from about 20 wt. % to 60 wt. % from about 25 wt. % to 50 wt. %, from about 30 wt. % to 40 wt. %, or from about 30 wt. % to 35 wt. %, based on the total weight of the organic components of the crude product composition.

The amount of 1,1,1-trifluoroethane (HFC-143a) and trifluoroethylene (HFO-1123) in the crude product composition, if present, in a combined amount of less than 2000 ppm, less than 1500 ppm, less than 1000 ppm, less than 700 ppm, less than 500 ppm, less than 100 ppm, less than 50 ppm, or less than 20 ppm, and for each of the foregoing, greater than or equal to 0 ppm, for example, based on total weight of organic components of the reactant composition.

The amount of 1,1,2-trifluoroethane (HFC-143) in the crude product composition, if present, may be less than 1.00 wt. %, less than 0.88 wt. %, less than 0.75 wt. %, or less than 0.50 wt. %, and for each of the foregoing, greater than or equal to 0.01 wt. %, for example, based on total weight of organic components of the reactant composition.

The amount of 1,1-difluoroethane (HFC-152a) in the crude product composition, if present, may be less than 1.50 wt. %, less than 1.00 wt. %, less than 0.88 wt. %, less than 0.75 wt. %, or less than 0.50 wt. %, and for each of the foregoing, greater than or equal to 0.01 wt. %, for example, based on total weight of organic components of the reactant composition.

The crude product composition from isomerization reactor can be further distilled to yield a purified product composition comprising, consisting essentially of, or consisting of, the trans-1,2-difluoroethylene (HFO-1132E) isomer in high purity, for example at least about 95 wt. %, at least about 99.0 wt. %, at least about 99.9 wt. %, at least about 99.99 wt. % or greater, based on total weight of organic components of the purified product composition.

The isomerization reaction may be conducted in any suitable reaction vessel or reactor, but it should preferably be constructed from materials which are resistant to corrosion including but not limited to nickel and its alloys, including Hastelloy (for example, Hastelloy C276), Inconel (for example Inconel 600), and Monel wherein the vessels which may be lined with fluoropolymers. These may be single pipe or multiple tubes packed with an isomerization catalyst.

Process 3—Step (ii)—Reaction Conditions

The temperature range for the isomerization reaction may be as low as about 100 °C, about 150 °C, about 200 °C, about 250 °C, about 300 °C, about 350 °C, about 400 °C, or as high as about 490 °C, about 550 °C, about 550 °C, about 600 °C, about 650 °C, about 700 °C, about 750 °C, about 800 °C or within any range encompassed by two of the foregoing values as endpoints, for example from about 100 °C to about 800 °C, from about 150 °C to about 750 °C, from about 200 °C to about 700 °C, from about 250 °C to about 650 °C, from about 300 °C to about 600 °C, from about 350 °C to about 550 °C, or from about 400 °C to about 500 °C.

The reaction may be conducted at atmospheric pressure, super-atmospheric pressure or under vacuum. The vacuum pressure can be from about 5 psig, about 10 psig, about 25 psig, about 50 psig, about 100 psig, about 150 tpsigorr, about 200 psig, about 250 psig, or any range using any of the foregoing values as endpoints, for example from about 5 psig to about 250 psig, about 10 psig to about 200 psig, about 25 psig to about 150 psig, or about 50 psig to about 100 psig.

Contact time of the reactants with the catalyst may range from about 0.5 seconds, about 1 second, about 5 seconds, about 10 seconds, to about 20 seconds, about 30 seconds, about 60 seconds, or about 120 seconds, or any range using any of the foregoing values as endpoints, for example from about 0.5 seconds to about 120 seconds, about 1 second to about 60 seconds, about 5 seconds to about 30 seconds, or about 10 seconds to about 20 seconds. However, longer or shorter times can be used. Shorter contact times yield lower amounts of impurities and longer contact times at lower temperatures may also yield lower amounts of impurities.

The reaction may also be conducted in an inert atmosphere substantially in the absence of oxygen. For example, the amount of oxygen present during the reaction may be less than 15 wt. %, less than 10 wt. %, less than 5 wt. %, or less than 1 wt. %, or any range using any two of the foregoing values as endpoints, for example from about 1 wt. % to about 15 wt. %, or about 5 wt. % to about 10 wt. %, based on a total weight of the reactants in the reactor.

The reaction may also be conducted substantially in the absence of water. For example, the amount of water present during the reaction may be less than 5 wt. %, less than 1 wt. %, less than 0.5 wt. %, or less than 0.05 wt. %, or any range using any two of the foregoing values as endpoints, for example 0.05 wt. % to about 5 wt. %, or about 0.5 wt. % to about 1 wt. %, based on a total weight of the reactants in the reactor.

### EXAMPLES

### Example 1

### Hydrogenation Reaction of CFC-113 to Produce CTFE

This example describes the production of CTFE from R-113 and H₂ without using a recycle stream. The experimental set up is described above and shown in FIG. 1.

A continuous vapor phase reaction system that utilizes a tubular reactor filled with silicon carbide catalyst is fed with a 1:1 mol ratio of R-113 and H₂. The reaction pressure is 1 psig and the reaction temperature is 525 °C. The catalyst contact time is in the range of 1-5 s with a target of 3 s.

As measured at the exit of the reactor, conversion of R-113 is about 25% per pass. In addition to R-1113 (CTFE), the reaction products include R-1123, R-123a and R-133. Selectivity to R-1113 is 95%. Selectivities to other major reaction products, R-1123, R-123a and R-133, are 4%, 0.25% and 0.25% respectively, based on total amount of organic components of the composition. The composition of bottom stream (e.g., stream 110 in FIG. 1) exiting the separation unit (e.g., separation unit 108 in FIG. 1) is 100% R-113. The composition of top stream (e.g., stream 112 in FIG. 1) exiting the separation unit (e.g., separation unit 108 in FIG. 1) without a recycle stream is 6.36 wt % H₂, 2.77 wt % R-1123, 89.6 wt % R-1113, 0.24 wt % R-133, and 1% R-123a, based on total amount of organic components of the composition.

The table below provides the reactor inlet and outlet composition for a basis of 1000 lb/hr R-113.

TABLE 5
Reactant and product compositions of a single-
pass hydrogenation reaction of CFC-113

| | |
|---|---|
| Reactor Feed | Reactor |

| | | Fresh R-113 | Fresh H2 | Outlet |
|---|---|---|---|---|
| H2 | lb/hr | 0 | 10.76 | 8.50 |
| HCI | lb/hr | 0 | 0 | 79.68 |
| R-1123 | lb/hr | 0 | 0 | 3.50 |
| R-1113 | lb/hr | 0 | 0 | 119.34 |
| R-133 | lb/hr | 0 | 0 | 0.32 |
| HF | lb/hr | 0 | 0 | 0 |
| R-123a | lb/hr | 0 | 0 | 0.41 |
| R-113 | lb/hr | 1000 | 0 | 799.00 |

### Example 2

This example describes the production of CTFE from R-113 and H₂ with a recycle stream. The experimental set up is described above and shown in FIG. 1.

The feed and reaction conditions are the same as Example 1. The table below provides the reactor inlet and outlet composition for a basis of 1000 lb/hr R-113. The composition of bottom stream (e.g., stream 110 in FIG. 1) exiting the separation unit (e.g., separation unit 108 in FIG. 1) is 90% R-113, based on total amount of organic components of the composition. The composition of top stream (e.g., stream 112 in FIG. 1) exiting the separation unit (e.g., separation unit 108 in FIG. 1) with a recycle stream is 6.44 wt % H₂, 2.65 wt % R-1123, 90.36 wt % R-1113, 0.24 wt % R-133, and 0.31% R-123a, based on total amount of organic components of the composition. The composition of purified product stream 116 exiting the hydrogen recovery unit (e.g., hydrogen recovery unit 114) includes at least 99.78 wt % R-1113, and less than or equal to 0.05 wt % R-1122, 0.05 wt % R-133, 0.095 wt % dimers, 0.05 wt % R-123a, and 0.05 wt % R-1123, based on total amount of organic components of the composition.

TABLE 6
Reactant and product compositions of a hydrogenation
reaction of CFC-113 with a recycle stream

| | | Reactor Feed | | | | |
|---|---|---|---|---|---|---|
| | | Fresh | Recycle | Fresh | Recycle | Reactor |
| | | R-113 | R-113 | H2 | H2 | Outlet |
| H2 | lb/hr | 0 | 0 | 2.25 | 8.49 | 8.49 |
| HCl | lb/hr | 0 | 0 | 0 | 0.00 | 79.69 |
| R-1123 | lb/hr | 0 | 0 | 0 | 0.19 | 3.70 |
| R-1113 | lb/hr | 0 | 0.02 | 0 | 0.26 | 119.61 |
| R-133 | lb/hr | 0 | 0.001 | 0 | 0.002 | 0.32 |
| HF | lb/hr | 0 | 0.0003 | 0 | 0 | 0.0003 |
| R-123a | lb/hr | 0 | 0.95 | 0 | 0 | 1.36 |
| R-113 | lb/hr | 203.55 | 795.00 | 0 | 1.44 | 799.00 |

### Example 3

### Hydrogenation Reaction of CFC-113 to Produce HFC-143

This example shows conversion of pure CFC-113 to HFC-143 using 4% Pd/C catalyst at temperatures ≥250 °C. The experimental set up is described above and shown in FIG. 1. The organic feed rate was 10 g/h, H₂ feeds rate was 150 ml/min, catalyst volume was 50 ml, and pressure was 45 psig.

Table 7 shows the GC-FID area percentages of the product stream after the reactor (e.g., stream 122 after reactor 120 as shown in FIG. 1) based on total amount of organic components of the product stream. CFC-113 conversion was 100% at all temperatures, based on total moles of organic components of the composition. The total of recyclable reaction intermediates (e.g., HCFC-133b and HCFC-133)/HCFC-123a formed as a function of temperature were 23.93% at 251 °C, 22.78% at 256 °C, and 20.21% at 260 °C, based on total moles of organic components of the composition. Less of reaction intermediates are generated at higher temperatures. It appears that the reaction intermediates can be further reacted at temperatures >250

TABLE 7
Conversion of pure CFC-113 using 4% Pd/C catalysts

| | GC-FID percent area | | | | | |
|---|---|---|---|---|---|---|
| | | | | | | Total |
| Temp/°C | R-143 | R-133b | R-133 | R-123a | R-113 | desired |
| 251 | 70.89 | 18.65 | 2.04 | 3.24 | 0.00 | 94.81 |
| 256 | 71.64 | 18.62 | 2.02 | 2.14 | 0.00 | 94.42 |
| 260 | 73.46 | 17.41 | 1.91 | 1.20 | 0.00 | 93.99 |

Reaction product stream 122 is sent through separation unit 124 to remove and/or recover streams comprising H₂, HCl, HF and a stream comprising 143 with the composition as shown in Table 8.

TABLE 8
Composition of final product stream after conversion of pure
CFC-113 using 4% Pd/C catalysts and post processing
GC-FID percent area

| R-143 | R-133b | R-133 | R-123a | R-113 | Others |
|---|---|---|---|---|---|
| 99.0 | 0.5 | 0.05 | 0.03 | 0.00 | 0.42 |

### Examples 4-8

### Hydrogenation Reaction of CFC-113 to Produce HCFC-123a

Examples 4-8 show the production of R-123a from R-113 and H₂. The experimental set up is described above and shown in FIG. 2. Reaction conditions were set to 450 °F (232 °C), 90 psia pressure, and 30 second contact time using 4% Pd/C catalyst.

Examples 4-8 show the conversion of CFC-113 to useful products and intermediates illustrating the effect of H₂:R-113 mole ratio to reaction product distribution. The results are presented in Table 9 and Table 10 and may be utilized in a production plant for optimization based on product demand. For example, if the product demand is 1113 with little or no simultaneous 143 demand, the process may be adjusted to operate with hydrogen to CFC-113 mole ratio of 1 or less. If the production of 143 is necessary, the hydrogen to CFC-113 mole ratio may be adjusted based on the desired product ratio. The composition of bottom stream (e.g., stream 210) from separation unit (e.g., separation unit 208) includes 80-100 wt % R-123a, 0.01-1 wt % R-142 isomers, 0.01-15 wt % R-133, and 0-0.5 wt % R-113, based on total amount of organic components of the composition.

TABLE 9
Conversion of CFC-113 to HFC-123a and HFC-
143 with different hydrogen molar ratio

| | Example | Example | Example | Example | Example |
|---|---|---|---|---|---|
| | 4 | 5 | 6 | 7 | 8 |
| H2:R-113 | 0.5:1 | 1:1 | 1.5:1 | 2:1 | 3:1 |
| Mol Ratio | | | | | |
| R-113 | 50% | 100% | 100% | 100% | 100% |
| Conversion | | | | | |
| Selectivity | 93.09% | 92.73% | 61.58% | 40.80% | 18.92% |
| to R-123a | | | | | |
| Selectivity | 6.91% | 7.27% | 0.96% | 0.26% | 0.04% |
| to CTFE | | | | | |
| Selectivity | 0% | 0% | 9.18% | 25.76% | 51.36% |
| to R-143 | | | | | |
| Selectivity | 0% | 0% | 28.28% | 33.17% | 29.68% |
| to other | | | | | |
| components | | | | | |
| (see | | | | | |
| Table 10) | | | | | |

TABLE 10
Composition of other components (mol %) in Examples 6-8

| Other | | | |
|---|---|---|---|
| Components | Example 6 | Example 7 | Example 8 |
| R-133 | 4.52% | 4.00% | 2.83% |
| R-133b | 23.31% | 27.26% | 19.58% |
| Ethane | 0.00% | 0.00% | 0.12% |
| R-152a | 0.23% | 1.28% | 5.60% |
| R-142b | 0.11% | 0.25% | 0.27% |
| R-142 | 0.05% | 0.10% | 0.15% |
| R-160 | 0.01% | 0.06% | 0.29% |
| R-1123 | 0.01% | 0.01% | 0.00% |
| R-143a | 0.00% | 0.04% | 0.13% |
| HF | 0.45% | 2.03% | 8.12% |
| R-152 | 0.03% | 0.14% | 0.52% |
| R-161 | 0.00% | 0.03% | 0.17% |

### Example 9

### Vapor Phase Dehydrochlorination Reaction of HCFC-123a

This example shows production of CTFE from HCFC-123a using vapor phase reaction. The experimental set up is described above and shown in FIG. 2.

About 40 ml of 10% CsCl/MgF₂ catalyst was charged into a ¾×0.035″ tube Inconel 625 reactor. The reactor was installed in the middle of an electric 3-zone split furnace. Process temperatures were recorded using a multi-point thermocouple placed inside the reactor and within the catalyst bed. The reactor was firstly heated to desired temperatures in nitrogen flow, and then a stream containing 94.6% pure HCFC-123a was fed into the bottom of the vertically mounted reactor at a feed rate of 12 g/h to start a reaction. The reactor pressure was set at 1 atm. The reactor effluent was periodically sampled and analyzed by GC-MS and GC for its compositions to determine raw material conversion level and product selectivity.

As shown in Table 11, both HCFC-123a conversion and CTFE selectivity were increased with increasing temperature over the 10% CsCl/MgF₂ catalyst. At 500 °C, on average, the HCFC-123a conversion and CFO-1113 selectivity were 16.2 and 96.7%, respectively. As a comparison, the empty Inconel 625 reactor provided a conversion of about 6% and a CFO-1113 selectivity of about 94% at the same 500 °C. The composition of purified product stream 236a exiting the separation unit (e.g., separation unit 234 in FIG. 2a) includes at least 99.78 wt % R-1113, and less than or equal to 0.05 wt % R-1122, 0.05 wt % R-133, 0.095 wt % dimers, 0.05 wt % R-123a, and 0.05 wt % R-1123, based on total amount of organic components of the composition.

TABLE 11
Conversion of HCFC-123a to CTFE using vapor phase reaction

| | | R-123a conversion | |
|---|---|---|---|
| Catalyst | T (°C) | (%) | CTFE selectivity (%) |
| 10% CsCl/MgF₂ | 450 | 5.0 | 77.2 |
| 10% CsCl/MgF₂ | 480 | 8.8 | 82.9 |
| 10% CsCl/MgF₂ | 500 | 16.2 | 96.7 |
| Inconel 625 | 500 | 6.5 | 93.9 |
| (empty tube) | | | |

### Example 10

### Liquid Phase Dehydrochlorination Reaction of HCFC-123a

This example shows production of CTFE from HCFC-123a using liquid phase reaction. The experimental set up is described above and shown in FIG. 2.

A reaction for converting HCFC-123a to CFO-1113 was carried out in a system having a single reactor. The reactor was a continuous stirred reactor. The reaction was conducted continuously for 52 hours. During the experiment, the temperature in the reactor varied between about 50 °C and about 60 °C. The pressure in the reactor varied between about 30 psig and 113 psig.

During the reaction, an organic feed stream containing HCFC-123a was provided to the reactor at a flow rate of about 5 ml/min. Gas chromatography area percents of the organic feed revealed that the organic feed contained about 0.81% HFO-1113, about 95% HCFC-123a, and about 3.2% CFC-113.

During the reaction, a KOH feed stream was provided to the reactor at a flow rate of about 12 ml/min. Based upon the feed rates of the organic feed stream and the KOH feed stream, the molar ratio of KOH to HCFC-123a was calculated to be about 1, or slightly above 1. The theoretical residence time in the reactor during the reaction was calculated to be about 2 hours.

A collection vessel was used to collect spent KOH and any organic that was carried out with spent KOH. MeCl2 was added inside the collection vessel in order to trap organic. The volume of MeCl2 inside the cylinder was 20% of the total volume of liquid in collection vessel. Product and un-reacted organic was collected in collection vessel that were held in dry ice. Vapor samples were taken once an hour, and liquid samples were taken at longer intervals.

The overall mass balance of the reaction was calculated to be about 82% for HCFC-123a and about 87% for KOH. The average single pass conversion was calculated to be about 39%. Selectivity to CFO-1113 was calculated to be about 90%. The composition of purified product stream 236b exiting the separation unit (e.g., separation unit 234 in FIG. 2b) includes at least 99.78 wt % R-1113, and less than or equal to 0.05 wt % R-1122, 0.05 wt % R-133, 0.095 wt % dimers, 0.05 wt % R-123a, and 0.05 wt % R-1123, based on total amount of organic components of the composition.

### Example 11

### Hydrogenation Reaction of HCFC-123a to Produce HFC-143

This example shows the production of R-143 from R-123a and H₂. The experimental set up is described above and shown in FIG. 2. Reaction conditions were set to 450 °F (232 °C), 90 psia pressure, and 12 second contact time using 4% Pd/C catalyst. The feed stream for Example 11 includes substantially pure (>99%) R-123a. As shown in Table 12 and Table 13 below, the reaction achieves a conversion of R-123a of 69% and a selectivity to R-143 of 54.6%.

TABLE 12
Conversion of HCFC-123a to HFC-143 using vapor phase reaction

| | |
|---|---|
| H2:R-123a Mol Ratio | 3:1 |
| R-123a Conversion | 69% |
| Selectivity to R-143 | 54.6% |
| Selectivity to R-133b | 36.9% |
| Selectivity to R-152a | 4.4% |
| Selectivity to others (see | 4.1% |
| table below) | |

TABLE 13
Conversion of HCFC-123a to HFC-143 using vapor phase reaction

| Component | Selectivity |
|---|---|
| R-133 | 2.61% |
| R-1123 | 0.002% |
| R-143a | 0.02% |
| R-142b | 0.31% |
| Ethane | 0.07% |
| R-160 | 0.40% |
| CTFE | 0.02% |
| 142 | 0.12% |
| 152 | 0.46% |
| 161 | 0.11% |

When the conversion of R-123a is 69%, the composition of the stream (e.g., stream 222 in FIG. 2a or 2b) including the second product composition is shown in Table 14 below.

TABLE 14
Composition of Stream 222

| | | Basis with no R- | |
|---|---|---|---|
| | Organic Basis | 123A | Expected Range |
| | Outlet mol % | Outlet mol % | mol % |
| R-133 | 1.797% | 2.607% | 0-5% |
| R-133b | 25.458% | 36.925% | 30-40% |
| R-1123 | 0.001% | 0.002% | |
| R-143 | 37.639% | 54.591% | 50-60% |
| R-152a | 3.009% | 4.365% | 0-5% |
| R-143a | 0.016% | 0.024% | 0-0.05% |
| R-142b | 0.216% | 0.314% | 0-0.5% |
| R-123a | 31.054% | | |
| Ethane | 0.045% | 0.066% | 0-0.1% |
| R-160 | 0.273% | 0.396% | 0-0.5% |
| CTFE (R-1113) | 0.017% | 0.025% | 0-0.05% |
| R-142 | 0.084% | 0.122% | 0-0.25% |
| R-152 | 0.316% | 0.459% | 0-1% |
| R-161 | 0.073% | 0.106% | 0-0.25% |

### Example 12

### Hydrogenation Reaction of CFC-113 to Produce CTFE

This example describes the production of CTFE from R-113 and H₂ without using a recycle stream. The experimental set up is described above and shown in FIG. 1.

The feed and reaction conditions are the same as Example 1, except using the catalyst and support combinations as shown in Table 15 below. Similar results and compositions are observed for all catalyst and support combinations shown in Table 15.

TABLE 15
Catalyst and Support Selection

| Catalyst | Support |
|---|---|
| Silicon carbide | Activated Carbon |
| Silicon nitride | Activated Carbon |
| Silicon oxide | Activated Carbon |
| Silicon carbide | None |
| Silicon nitride | None |
| Silicon oxide | None |

### Example 13

This example describes the production of CTFE from R-113 and H₂ with a recycle stream. The experimental set up is described above and shown in FIG. 1.

The feed and reaction conditions are the same as Example 2, and similar results are measured for all combinations of catalyst and support shown in Table 15. Similar results and compositions are observed for all catalyst and support combinations shown in Table 15.

### Example 14

### Hydrogenation Reaction of CFC-113 to Produce CTFE

This example describes the production of CTFE from R-113 and H₂ without using a recycle stream. The experimental set up is described above and shown in FIG. 1.

The feed and reaction conditions are the same as Example 1, except using the catalyst and support combinations as shown in Table 16 below. Similar results and compositions are observed for all catalyst and support combinations shown in Table 16.

TABLE 16
Catalyst and Support Selection

| Catalyst | Support |
|---|---|
| Pd | Activated Carbon |
| Pd | alpha(α)-Al₂O₃ |
| Pd | theta(θ)-Al₂O₃ |
| Pd | delta(δ)-Al₂O₃ |
| Pd | gamma(γ)-Al₂O₃ |
| Pt | Activated Carbon |
| Pt | alpha(α)-Al₂O₃ |
| Pt | theta(θ)-Al₂O₃ |
| Pt | delta(δ)-Al₂O₃ |
| Pt | gamma(γ)-Al₂O₃ |

### Example 15

This example describes the production of CTFE from R-113 and H₂ with a recycle stream. The experimental set up is described above and shown in FIG. 1.

The feed and reaction conditions are the same as Example 2, and similar results and compositions are observed for all combinations of catalyst and support shown in Table 16.

### Example 16

This example describes the production of E and Z-1,2-difluoroethylene (HFO-1132 Z/E) from 1,1,2-trifluoroethane (HFC-143).

HFC-143 is produced according to Example 3. The experimental setup includes a 2-gallon Hastelloy C pressure vessel (Parr Instruments). The vessel is equipped with an agitator. The exit of reactor enters into a distillation column (1″OD, 18″ length). The product of the distillation goes into product collection cylinder (PCC). Reaction is performed in batch mode. The reactor is loaded with the 1600 grams of organic feed. The reactor is heated to 116 °C with stirrer (600 rpm) on. When the reactor reaches temperature and temperature is stable, 2640 g of 45% KOH solution is added over three hours using Eldex pump. After complete addition of KOH solution, the reaction mixture is kept at 116 °C under stirring for an additional 0.5 hours. Then the reactor exit is opened to distillation column. Due to fluctuations in temperature, system is allowed to equilibrate for 2 hours (total reflux). Then the hold time of 10 hours is started. At the end of the hold time, the reactor temperature is reduced to 70 °C and the reactor content is opened to a dry ice cooled product collection cylinder (PCC). About 1317 g of the organic product is collected in the PCC. The overall mass balance is 98.7%.

The organic feed and product are analyzed using gas chromatography, results are shown in Table 17. It is assumed that the GC-FID area % are equal to weight %, and conversion and selectivity values were calculated using the equations shown above. Results are presented in Table 18.

TABLE 17
GC-FID area % of the organic feed and product

| Molecule | Feed | Product |
|---|---|---|
| R143 | 99.57 | 74.00 |
| R133s | 0.28 | 0.00 |
| R123a | 0.00 | 0.00 |
| R152a | 0.00 | 0.00 |
| R1113 | 0.00 | 0.00 |
| R1123 | 0.00 | 0.22 |
| R1132Z | 0.00 | 22.20 |
| R1132E | 0.00 | 1.40 |
| R1132a | 0.00 | 1.83 |
| Other | 0.16 | 0.34 |

TABLE 18
Reaction conditions and the corresponding conversion/selectivity values

| | Mole | | | | | | | |
|---|---|---|---|---|---|---|---|---|
| | mole | | | | | | | |
| | ratio | | | | | | | |
| | of | | Conv | Selec. | Selec. | Selec. | Selec. | |
| | KOH | KOH | of | to | to | To | to | |
| | to | Weight | 143. | 1132Z | R1132E | 1132a | Heavies | Yield, |
| T, °C | Org* | %, % | (%) | (%) | (%) | (%) | (%) | %** |
| 116 | 1.1 | 45 | 39 | 62 | 4 | 5 | 29 | 25.6 |

*n(KOH)/n(R143 + R133b + R133 + 123a) mole ratio.
**Yield is defined as moles of 1132Z and 1132E made divided by initial amount of 143.

### Example 17

This example describes the production of E-1,2-difluoroethylene (HFO-1132 E) from a mixture of E and Z-1,2-difluoroethylene (HFO-1132 Z/E) isomers.

The HFO-1132Z/E isomers produced in Example 16 above are further reacted with a catalyst to produce HFO-1132E using Step (ii), as described above. Table 19 shows the feed composition for the isomerization reaction in the order each component appears in the gas chromatogram.

TABLE 19
Isomerization Reaction Feed Component Concentrations

| Component name | GC-FID area % | Conc. (ppm) |
|---|---|---|
| HFC-143a | <0.0000 | <1 |
| HFO-1123 | 0.0005 | 5 |
| HFO-1132(E) | 0.0079 | 79 |
| HFC-152a | 0.4218 | 4218 |
| HFO-1132(Z) | 98.8897 | 988897 |
| HFC-143 | 0.4830 | 4830 |
| Other | 0.1970 | 1970 |

After running the isomerization reaction using the above feed, different concentrations of CTFE co-feed as the catalyst, and nitrogen gas a diluent, a product composition as shown below is obtained. Reactions are performed at 490 °C and 30 psig in an empty ¾″ Inconel tube with about 14-25 seconds residence time. Reactions are performed in the presence of different CTFE weight percentages ranging from 4 wt. % to 13 wt. %. The 1132Z conversion and selectivity toward 1132E in the isomerization reaction is a function of CTFE concentration.

Table 20 below shows the chemical composition of the crude product that is collected during parametric studies (different CTFE concentrations and residence time) in the order they appear in the gas chromatogram. Note that this material is not originated from single run conditions and the presented compositions should not be used to assess the isomerization reaction efficiency. However, the presented example shows how certain impurities impact the quality of final 1132E product. For example, some impurities such as 1132a and 1141 are separable from 1132E while other impurities such as 143a and 1123 are not separable.

TABLE 20
Crude Product Composition Following Isomerization

| Component name | GC-FID area % | Conc. (ppm) |
|---|---|---|
| HFO-1132a | 0.0052 | 52 |
| HFC-143a | 0.0022 | 22 |
| HFO-1141 | 0.0243 | 243 |
| HFO-1123 | 0.0005 | 5 |
| HFO-1132(E) | 20.4650 | 204650 |
| HFC-152a | 1.4295 | 14295 |
| HFO-1132(Z) | 65.2216 | 652216 |
| HCFO-1113 | 7.8746 | 78746 |
| HFC-143 | 0.8753 | 8753 |
| Other | 4.0975 | 40975 |

Distillation of the crude product in Table 20 is performed to form a purified product composition. After distillation of the above crude product composition, a product with the below purity profiles is obtained, as shown in Table 21.

TABLE 21
Purified Product Composition Following Distillation

| Component name | GC-FID area % | Conc. (ppm) |
|---|---|---|
| HFC-32 | 0.0010 | 10 |
| HFC-143a | 0.0089 | 89 |
| HFO-1141 | 0.0005 | 5 |
| HFO-1123 | 0.0095 | 95 |
| HFO-1132(E) | 99.9746 | 999746 |
| HFC-152a | 0.0000 | 0 |
| HFO-1132(Z) | 0.0023 | 23 |
| HCFO-1113 | 0.0018 | 18 |
| HFC-143 | 0.0002 | 2 |
| Other | 0.0013 | 13 |

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

### ASPECTS

Aspect 1 is a method for producing chlorotrifluoroethylene (CTFE) and 1,1,2-trifluoroethane (HFC-143), comprising: reacting 1,1,2-trichloro-1,2,2-trifluoroethane (CFC-113) with hydrogen in the presence of a first catalyst at a temperature from about 450 °C to about 600 °C to produce a first product composition comprising CTFE and CFC-113; separating at least some of the CTFE from the first product composition to produce a first processed composition and a first purified product composition; and further reacting the first processed composition comprising CFC-113 with hydrogen in the presence of a second catalyst at a temperature of from 150 °C to 400 °C to produce a second product composition comprising HFC-143.

Aspect 2 is the method of Aspect 1, wherein the first catalyst is a silicon-based catalyst selected from silicon carbide, silicon nitride, and silicon oxide.

Aspect 3 is the method of Aspect 2, wherein the first catalyst is supported on a carbon support.

Aspect 4 is the method of Aspect 2, wherein the first catalyst is unsupported.

Aspect 5 is the method of any one of Aspects 1-4, wherein the first reacting step comprises contacting CFC-113 with the first catalyst for a duration of from about 0.1 second to about 6 seconds.

Aspect 6 is the method of any one of Aspects 1-5, wherein the molar ratio of hydrogen to CFC-113 in the first reacting step is from about 1:10 to about 1:1.

Aspect 7 is the method of any one of Aspects 1-6, wherein the first purified product composition comprises: 90 wt % to 99.95 wt % chlorotrifluoroethylene (CTFE); 0.01 wt % to 2 wt % 2-chloro-1,1-difluoroethylene (HCFC-1122); 0.01 wt % to 2 wt % 1-chloro-1,2,2-trifluoroethane (HCFC-133); 0.01 wt % to 2 wt % dimers; 0.01 wt % to 2 wt % 1,2-dichloro-1,1,2-trifluoroethane (HCFC-123a); and 0.01 wt % to 2 wt % trifluoroethylene (HFO-1123), based on the total amount of organic components in the first processed composition.

Aspect 8 is the method of any one of Aspects 1-7, wherein the second catalyst is palladium metal supported on a carbon support.

Aspect 9 is the method of any one of Aspects 1-8, further comprising reacting the second product composition to create at third product composition comprising HFC-1132E.

Aspect 10 is a method for producing chlorotrifluoroethylene (CTFE) and 1,1,2-trifluoroethane (HFC-143), comprising: reacting 1,1,2-trichloro-1,2,2-trifluoroethane (CFC-113) with hydrogen in the presence of a first catalyst at a temperature from about 150 °C to 400 °C to produce a first product composition comprising 1,2-dichloro-1,1,2-trifluoroethane (HCFC-123a); and at least one of: reacting HCFC-123a with hydrogen in the presence of a second catalyst at a temperature from about 150 °C to 400 °C according to a first reaction pathway to produce a second product composition comprising 1,1,2-trifluoroethane (HFC-143); or reacting HCFC-123a in a vapor phase or a liquid phase dehydrochlorination reaction according to a second reaction pathway to produce a third product composition comprising CTFE.

Aspect 11 is the method of Aspect 10, wherein the first catalyst is palladium metal supported on a carbon support.

Aspect 12 is the method of Aspects 10 or 11, wherein the second catalyst is palladium metal supported on a carbon support.

Aspect 13 is the method of any one of Aspects 10-12, wherein the first reacting step comprises contacting CFC-113 with the first catalyst for a duration of from about 1 second to about 60 seconds.

Aspect 14 is the method of any one of Aspects 10-13, wherein the molar ratio of hydrogen to CFC-113 in the first reacting step is from about 3:1 to about 10:1.

Aspect 15 is the method of any one of Aspects 10-14, further comprising: separating HFC-143 from the first product composition to produce a first processed composition.

Aspect 16 is the method of Aspect 15, wherein the first processed composition comprises: 80 wt % to 99.97 wt % 1,2-dichloro-1,1,2-trifluoroethane (HCFC-123a); 0.01 wt % to 3 wt % HCFC-142 isomers comprising 1-chloro-1,2-difluoroethane (HCFC-142a), 1-chloro-2,2-difluoroethane (HCFC-142), and 1-chloro-1,1-difluoroethane (HCFC-142b); 0.01 wt % to 15 wt % 1-chloro-1,2,2-trifluoroethane (HCFC-133); and 0.01 wt % to 2 wt % 1,1,2-trichloro-1,2,2-trifluoroethane (CFC-113), based on the total amount of organic components in the first processed composition.

Aspect 17 is the method of any one of Aspects 10-16, wherein the second reaction pathway is a vapor phase dehydrochlorination reaction comprising reacting HCFC-123a in the presence of a third catalyst at a temperature of from about 400 °C to about 600 °C.

Aspect 18 is the method of Aspect 17, wherein the third catalyst comprises a metal halide, a halogenated metal oxide, a zero-valent metal, or a zero-valent metal alloy.

Aspect 19 is the method of Aspect 18, wherein the third catalyst comprises a mixture of CsCl and MgF₂.

Aspect 20 is the method of any one of Aspects 10-16, wherein the second reaction pathway is a liquid phase dehydrochlorination reaction comprising reacting HCFC-123a in the presence of a base at a temperature of from about 40 °C to about 100 °C.

Aspect 21 is the method of Aspect 20, wherein the base comprises KOH, NaOH, CaO, or Ca(OH)₂.

Aspect 21 is the method of Aspects 19 or 20, wherein the molar ratio of the base to HCFC-123a in the liquid phase dehydrochlorination reaction is from about 0.5:1 to about 3:1.

Aspect 22 is the method of any one of Aspects 10-21, further comprising reacting the second product composition to create at third product composition comprising HFC-1132E.

Aspect 23 is a product composition comprising HFC-1132E produced according to the method of Aspect 22.

What is claimed is:

1. A method for producing chlorotrifluoroethylene (CTFE) and 1,1,2-trifluoroethane (HFC-143), comprising:
reacting 1,1,2-trichloro-1,2,2-trifluoroethane (CFC-113) with hydrogen in the presence of a first catalyst at a temperature from about 450 °C to about 600 °C to produce a first product composition comprising CTFE and CFC-113;
separating at least some of the CTFE from the first product composition to produce a first processed composition and a first purified product composition; and
further reacting the first processed composition comprising CFC-113 with hydrogen in the presence of a second catalyst at a temperature of from 150 °C to 400 °C to produce a second product composition comprising HFC-143.

2. The method of claim 1, wherein the first catalyst is a silicon-based catalyst selected from silicon carbide, silicon nitride, and silicon oxide.

3. The method of claim 2, wherein the first catalyst is supported on a carbon support.

4. The method of claim 2, wherein the first catalyst is unsupported.

5. The method of claim 1, wherein the first reacting step comprises contacting CFC-113 with the first catalyst for a duration of from about 0.1 second to about 6 seconds.

6. The method of claim 1, further comprising reacting the second product composition to produce a third product composition comprising HFC-1132E.

7. The method of claim 1 wherein the first purified product composition comprises:
90 wt % to 99.95 wt % chlorotrifluoroethylene (CTFE);
0.01 wt % to 2 wt % 2-chloro-1,1-difluoroethylene (HCFC-1122);
0.01 wt % to 2 wt % 1-chloro-1,2,2-trifluoroethane (HCFC-133);
0.01 wt % to 2 wt % dimers;
0.01 wt % to 2 wt % 1,2-dichloro-1,1,2-trifluoroethane (HCFC-123a); and
0.01 wt % to 2 wt % trifluoroethylene (HFO-1123), based on the total amount of organic components in the first processed composition.

8. The method of claim 1, wherein the second catalyst is palladium metal supported on a carbon support.

9. A method for producing chlorotrifluoroethylene (CTFE) and 1,1,2-trifluoroethane (HFC-143), comprising:
reacting 1,1,2-trichloro-1,2,2-trifluoroethane (CFC-113) with hydrogen in the presence of a first catalyst at a temperature from about 150 °C to 400 °C to produce a first product composition comprising 1,2-dichloro-1,1,2-trifluoroethane (HCFC-123a); and
at least one of:
reacting HCFC-123a with hydrogen in the presence of a second catalyst at a temperature from about 150 °C to 400 °C according to a first reaction pathway to produce a second product composition comprising 1,1,2-trifluoroethane (HFC-143); or
reacting HCFC-123a in a vapor phase or a liquid phase dehydrochlorination reaction according to a second reaction pathway to produce a third product composition comprising CTFE.

10. The method of claim 9, wherein at least one of the first catalyst and the second catalyst is palladium metal supported on a carbon support.

11. The method of claim 9, wherein the first reacting step comprises contacting CFC-113 with the first catalyst for a duration of from about 1 second to about 60 seconds.

12. The method of claim 9, wherein the molar ratio of hydrogen to CFC-113 in the first reacting step is from about 3:1 to about 10:1.

13. The method of claim 9, further comprising:
separating HFC-143 from the first product composition to produce a first processed composition.

14. The method of claim 13, wherein the first processed composition comprises:
80 wt % to 99.97 wt % 1,2-dichloro-1,1,2-trifluoroethane (HCFC-123a);
0.01 wt % to 3 wt % HCFC-142 isomers comprising 1-chloro-1,2-difluoroethane (HCFC-142a), 1-chloro-2,2-difluoroethane (HCFC-142), and 1-chloro-1,1-difluoroethane (HCFC-142b);
0.01 wt % to 15 wt % 1-chloro-1,2,2-trifluoroethane (HCFC-133); and
0.01 wt % to 2 wt % 1,1,2-trichloro-1,2,2-trifluoroethane (CFC-113), based on the total amount of organic components in the first processed composition.

15. The method of claim 9, wherein the second reaction pathway is a vapor phase dehydrochlorination reaction comprising reacting HCFC-123a in the presence of a third catalyst at a temperature of from about 400 °C to about 600 °C.

16. The method of claim 15, wherein the third catalyst comprises a metal halide, a halogenated metal oxide, a zero-valent metal, or a zero-valent metal alloy.

17. The method of claim 9, wherein the second reaction pathway is a liquid phase dehydrochlorination reaction comprising reacting HCFC-123a in the presence of a base at a temperature of from about 40 °C to about 100 °C.

18. The method of claim 17, wherein the base comprises KOH, NaOH, CaO, or Ca(OH)₂.

19. The method of claim 9, further comprising reacting the second product composition to produce a third product composition comprising HFC-1132E.

20. A product composition comprising HFC-1122E produced according to the method of claim 19.
